SEQUENCE LISTING (43 sequences)

SEQ ID NO: 1 - DNA, 8667 bp, synthetic construct
  source 1..8667: mol_type = other DNA
  misc_feature 1..8667: note = synthetic plasmid
aaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaagtttaaaccataatgcgtgacaccgccatgatggttgtattctaccaatgagacatggccgctgatcctgttgtgtgggtcatgggacatcacctcttgggggggattctcctataattggcaccgtgtatgcctcaaccactaacttccaccctataactgaatatattacataagcaaatctactttttgtttgtgttgatcgccatcgttgaaattcgcgcaacttctggtggctcaacgctgctgttctatcggtatcctaagagatgtctttgccctgagtctagggtaaactatccaccttcgttgctgtttgactagacagctactaactttacggtagtaaatgaataacggctcgctctcatgatcacttctctacatcaccctaacaagtgtattatttttttttcaggtgggtgttgctgttggtgctagcatatggcggccgcggatccctcgagtttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaatatctagataaaatgttaaagttattgtccttgatggtcccattagcttctgcagctgttatccacagacgtgatgctaacatttcagctattgcatccgaatggaactccacttctaactcttcttcatctttatctttaaacagaccagctgtccattattctccagaggaaggttggatgaacgacccaaacggtttatggtacgatgctaaagaggaagattggcacatctactatcaatactatcctgatgcccctcattggggtttgccattgacttggggtcatgcagtctccaaagatttgaccgtctgggacgaacaaggtgttgcattcggtccagagtttgaaacagcaggtgccttttctggttctatggttattgattacaataacacctccggtttctttaactcatccaccgacccaagacaacgtgtcgttgccatttggactttggattattctggctctgaaacacaacaattatcttattctcatgatggtggttatacattcaccgaatattctgacaaccctgtcttagatattgactcagacgcttttagagatccaaaggttttctggtatcaaggtgaagattccgaatcagaaggtaactgggtcatgacagttgccgaagcagatcgtttctccgtcttaatctactcttctccagaccttaagaattggaccttagaatcaaacttttccagagaaggctacttaggctataactatgagtgtcctggtttagttaaggtcccatacgtcaaaaacaccacatacgcatctgctccaggctcaaatatcacctcatctggtccacttcatccaaattctactgtttctttctcaaattcatcctctattgcatggaatgcttcttccgttccacttaacattactttatccaattctaccttggttgatgaaacttctcaattggaagaagttggttacgcatgggttatgattgtctcattcaatcctggctccattttaggcggttccggtactgaatacttcatcggtgactttaatggtacacacttcgagccacttgataagcaaactagattcttagatttgggtaaagattactacgctttgcaaactttcttcaataccccaaacgaggttgacgttttgggtatcgcatgggcctctaattggcaatatgctaaccaagttccaacagatccatggagatcatccatgtccttggttagaaacttcactatcactgaatacaacatcaattctaatactactgcattggtcttgaactctcaaccagttttagattttacctctttaagaaagaacggcacatcatatactttagagaatcttacattaaactcctcttctcacgaggttttggaatttgaagatcctaccggtgttttcgaattttcccttgaatattccgtcaactttaccggtattcacaactgggtttttaccgacttgtccttgtatttccaaggtgataaggattcagatgaatacttgagacttggttacgaagctaactccaagcagttctttttagatagaggtcattctaacattccatttgttcaagaaaatccattcttcactcagagactttcagtttccaatcctccatcctccaactcctccaccttcgatgtctacggtattgttgacagaaatatcattgaattgtatttcaacaatggtactgttacctctactaacacctttttcttctccactggtaacaatattggttccatcattgttaagtctggtgttgatgacgtctatgaaattgaatcattgaaggttaatcagttttacgttgactaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgctttaataaagtagtttgtttgtcaaggatggcgtcatacaaagaaagatcagaatcacacacttcccctgttgctaggagacttttctccatcatggaggaaaagaagtctaacctttgtgcatcattggatattactgaaactgaaaagcttctctctattttggacactattggtccttacatctgtctagttaaaacacacatcgatattgtttctgattttacgtatgaaggaactgtgttgcctttgaaggagcttgccaagaaacataattttatgatttttgaagatagaaaatttgctgatattggtaacactgttaaaaatcaatataaatctggtgtcttccgtattgccgaatgggctgacatcactaatgcacatggtgtaacgggtgcaggtattgtttctggcttgaaggaggcagcccaagaaacaaccagtgaacctagaggtttgctaatgcttgctgagttatcatcaaagggttctttagcatatggtgaatatacagaaaaaacagtagaaattgctaaatctgataaagagtttgtcattggttttattgcgcaacacgatatgggcggtagagaagaaggttttgactggatcattatgactccaggggttggtttagatgacaaaggtgatgcacttggtcaacaatatagaactgttgatgaagttgtaaagactggaacggatatcataattgttggtagaggtttgtacggtcaaggaagagatcctatagagcaagctaaaagataccaacaagctggttggaatgcttatttaaacagatttaaatgattcttacacaaagatttgatacatgtacactagtttaaataagcatgaaaagaattacacaagcaaaaaaaaaaaaataaatgaggtactttacgttcacctacaaccaaaaaaactagatagagtaaaatcttaagatttagaaaaagttgtttaacaaaggctttagtatgtgaatttttaatgtagcaaagcgataactaataaacataaacaaaagtatggttttctttatcagtcaaatcattatcgattgattgttccgcgtatctgcagatagcctcatgaaatcagccatttgcttttgttcaacgatcttttgaaattgttgttgttcttggtagttaagttgatccatcttggcttatgttgtgtgtatgttgtagttattcttagtatattcctgtcctgagtttagtgaaacataatatcgccttgaaatgaaaatgctgaaattcgtcgacatacaatttttcaaactttttttttttcttggtgcacggacatgtttttaaaggaagtactctataccagttattcttcacaaatttaattgctggagaatagatcttcaacgccccgggggatctggatccgcggccgcgagctctaatgattcaagaaaaagttcaaataaactaatggatcaacctatttcgaccctttcttcattgctacttcttccttaagcaacagatgattaagtagatactgtttttttagccaatagtatctcgccgaggagttatacttgactagctcttgctcaagaatcttcctaagacgtactagcctagcatagtaatctgtttgtttctgtattgtttgttctaactgttctacagtcattgaatcaatatctccaatgtcttcgacgttgacaactttcccccccttggcagcattctcttttttgttggaatacgacattaaagattccttgattttctgggtaccttcaatgaccattgagggattaaatttgatttctttgattttataatggtcggctattagctcttccacttcgtcatcatgatcatcagatatgtcacgttgccttttcaatttattaaaattgtttatcagtttattgtgatcttgtatcaattcattgcgtactcttttctcaatatcaaaagctattttcttcccgctagactcaaaatcaactctgaagtcattttctcgctggaattcatgtatttcatggattaattctctattgatattctcgtatgcatcctgtaaactgttgccgttgatattatgaaccgcctttaaatgtttcaataaggcatctgctctagtaaatgccttcagacattcaggtaataaacagtaaaatggcttctcggctgtatgcgtcctaatgtttaaaccgtcaaaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttcta

SEQ ID NO: 2 - DNA, 3234 bp, synthetic construct
  source 1..3234: mol_type = other DNA
  misc_feature 1..3234: note = synthetic polynucleotide
attcgccacattcgacacattcgctgcattcgccacaacgtcacgtgaggcatcgtagttttgagtttttagatctcctcgagtttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaatatctagataaaatgttaaagttattgtccttgatggtcccattagcttctgcagctgttatccacagacgtgatgctaacatttcagctattgcatccgaatggaactccacttctaactcttcttcatctttatctttaaacagaccagctgtccattattctccagaggaaggttggatgaacgacccaaacggtttatggtacgatgctaaagaggaagattggcacatctactatcaatactatcctgatgcccctcattggggtttgccattgacttggggtcatgcagtctccaaagatttgaccgtctgggacgaacaaggtgttgcattcggtccagagtttgaaacagcaggtgccttttctggttctatggttattgattacaataacacctccggtttctttaactcatccaccgacccaagacaacgtgtcgttgccatttggactttggattattctggctctgaaacacaacaattatcttattctcatgatggtggttatacattcaccgaatattctgacaaccctgtcttagatattgactcagacgcttttagagatccaaaggttttctggtatcaaggtgaagattccgaatcagaaggtaactgggtcatgacagttgccgaagcagatcgtttctccgtcttaatctactcttctccagaccttaagaattggaccttagaatcaaacttttccagagaaggctacttaggctataactatgagtgtcctggtttagttaaggtcccatacgtcaaaaacaccacatacgcatctgctccaggctcaaatatcacctcatctggtccacttcatccaaattctactgtttctttctcaaattcatcctctattgcatggaatgcttcttccgttccacttaacattactttatccaattctaccttggttgatgaaacttctcaattggaagaagttggttacgcatgggttatgattgtctcattcaatcctggctccattttaggcggttccggtactgaatacttcatcggtgactttaatggtacacacttcgagccacttgataagcaaactagattcttagatttgggtaaagattactacgctttgcaaactttcttcaataccccaaacgaggttgacgttttgggtatcgcatgggcctctaattggcaatatgctaaccaagttccaacagatccatggagatcatccatgtccttggttagaaacttcactatcactgaatacaacatcaattctaatactactgcattggtcttgaactctcaaccagttttagattttacctctttaagaaagaacggcacatcatatactttagagaatcttacattaaactcctcttctcacgaggttttggaatttgaagatcctaccggtgttttcgaattttcccttgaatattccgtcaactttaccggtattcacaactgggtttttaccgacttgtccttgtatttccaaggtgataaggattcagatgaatacttgagacttggttacgaagctaactccaagcagttctttttagatagaggtcattctaacattccatttgttcaagaaaatccattcttcactcagagactttcagtttccaatcctccatcctccaactcctccaccttcgatgtctacggtattgttgacagaaatatcattgaattgtatttcaacaatggtactgttacctctactaacacctttttcttctccactggtaacaatattggttccatcattgttaagtctggtgttgatgacgtctatgaaattgaatcattgaaggttaatcagttttacgttgactaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagcgagctcttaatgtttgttttctttttttgtcgtagaaatgtgctgtgagatcaggaaattgttcaagctgttcacg

SEQ ID NO: 3 - DNA, 6174 bp, synthetic construct
  source 1..6174: mol_type = other DNA
  misc_feature 1..6174: note = synthetic polynucleotide
  misc_difference 780: note = n is a, c, g, or t
aaacctcggtagtgcctaccaatttaatgtttgactccttgcgttttctcctgtcgcggacaaacggtgcggctcccccgatgattcacgtaataagccggagtcaaccacagaggtcccctatgactcaacaaggcctcgtagaaactcggcttctcggagaaagagtcttttctttttcactggaaaatatttttttttcctttatattcttttgaaccaaaatgtggctactataaaagtgcctttattccccagcttttctagcatgattgagtcaccttccacaatgagtcttctttattgttagtattgtgaatattatccgtgcagttttcaagaacgtaaatcaacagcagtgataataccttcaaacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgaattctgcagatatcggccggccccatggagatctcctcgagtttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaatatctagataaaatgttaaagttattgtccttgatggtcccattagcttctgcagctgttatccacagacgtgatgctaacatttcagctattgcatccgaatggaactccacttctaactcttcttcatctttatctttaaacagaccagctgtccattattctccagaggaaggttggatgaacgacccaaacggtttatggtacgatgctaaagaggaagattggcacatctactatcaatactatcctgatgcccctcattggggtttgccattgacttggggtcatgcagtctccaaagatttgaccgtctgggacgaacaaggtgttgcattcggtccagagtttgaaacagcaggtgccttttctggttctatggttattgattacaataacacctccggtttctttaactcatccaccgacccaagacaacgtgtcgttgccatttggactttggattattctggctctgaaacacaacaattatcttattctcatgatggtggttatacattcaccgaatattctgacaaccctgtcttagatattgactcagacgcttttagagatccaaaggttttctggtatcaaggtgaagattccgaatcagaaggtaactgggtcatgacagttgccgaagcagatcgtttctccgtcttaatctactcttctccagaccttaagaattggaccttagaatcaaacttttccagagaaggctacttaggctataactatgagtgtcctggtttagttaaggtcccatacgtcaaaaacaccacatacgcatctgctccaggctcaaatatcacctcatctggtccacttcatccaaattctactgtttctttctcaaattcatcctctattgcatggaatgcttcttccgttccacttaacattactttatccaattctaccttggttgatgaaacttctcaattggaagaagttggttacgcatgggttatgattgtctcattcaatcctggctccattttaggcggttccggtactgaatacttcatcggtgactttaatggtacacacttcgagccacttgataagcaaactagattcttagatttgggtaaagattactacgctttgcaaactttcttcaataccccaaacgaggttgacgttttgggtatcgcatgggcctctaattggcaatatgctaaccaagttccaacagatccatggagatcatccatgtccttggttagaaacttcactatcactgaatacaacatcaattctaatactactgcattggtcttgaactctcaaccagttttagattttacctctttaagaaagaacggcacatcatatactttagagaatcttacattaaactcctcttctcacgaggttttggaatttgaagatcctaccggtgttttcgaattttcccttgaatattccgtcaactttaccggtattcacaactgggtttttaccgacttgtccttgtatttccaaggtgataaggattcagatgaatacttgagacttggttacgaagctaactccaagcagttctttttagatagaggtcattctaacattccatttgttcaagaaaatccattcttcactcagagactttcagtttccaatcctccatcctccaactcctccaccttcgatgtctacggtattgttgacagaaatatcattgaattgtatttcaacaatggtactgttacctctactaacacctttttcttctccactggtaacaatattggttccatcattgttaagtctggtgttgatgacgtctatgaaattgaatcattgaaggttaatcagttttacgttgactaattaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcatagcctcatgaaatcagcgagctcgtctgatatttgctaaattgaaatgaaccttaccatgccacatctatagacatcaaaaccattttcaatttgtcgatatcttttgcatatcaaagtaataccaagcatgttcaaaaagaaaagaaagcataactttaatactctattcgaaacattccgatccacaacacattagtctttttaggcccgttgttcatctttctattactttattcctaactgtatttttataattccgggtttataaaagattaaactaatatagcgcattctttttgggtacaaacatacataacggaggttt

SEQ ID NO: 4 - DNA, 4983 bp, synthetic construct
  source 1..4983: mol_type = other DNA
  misc_feature 1..4983: note = synthetic polynucleotide
  misc_difference 531: note = n is a, c, g, or t
tcgacatagggcacaatagtcagcggggaaacggagttcccgagggtgtcggagatactccagctaattgacccgcgcgccccttaattaactcgagggatcccgggcccctggaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaatggcagtataccctatcaggtattcagatagttttatttttgtaggtatataatctgaagcctttgaactattttctcgtatatatcatggagtatacattgcattagcaacattgcatactagttcataacttcgtataatgtatgctatacgaagttattaattaacaagggcgaattctgcagatatcggccggccgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatgacacaaactttacaagccgcttttgaaaaaacaaagtctgaattgactgttacaactgaaaagttggaagaaatcacaaaccattttgttgaagaactcgaaaagggtatttctccagccggtggtaacattccaatgaatcctacttgggttatggattatccattaggttccgaaactggtgaatacttggcaatcgatttaggtggtaccaaccttagagtcatcagagtaacattacaaggtgattccaaattcaaatcagtcaatgaaaaatacccaattcctgttccgatgagaaccggtaataaagacgatctatttgatttcattgcaaagtctttagatgattttattaagcatcagtatggtgaggattacaaaggtgaaaaattgccattaggtttcaccttctcttaccctgcacatcaaaacgcaatcaatcatggtgtcttgcagacatggaccaaaggttttgatattccaggcgttgaagggcatgatgttgttccgatgttacaagcctcccttgataaattgaataccccagttgaggttgttgccttgattaatgatactaccggtactttagtcgcttccaaatattgtgatactgagaccatcatgggtttgatctttggtaccggttgtaatggtgcttattatgattttgcaaagaatattcctaaattagaaggtaagttagctagtgatatcaatgatgaaactccaatggctattaattgcgaatatggtgcttttgataacgattggaaagttctgccaagaaccaagtatgatatccaaattgaccaagaatctccaaggccaggtcagcaattctatgaaaaaatgattgctggttattatttaggtgaagttttaagattaattttattggactattattctcaaggactggttttcattaatcaggatatcaagtctttgcaagttccatttgcaatggatacctccttcccttcaatgattgaagaacatggtcctgaatacgctggaagattattcaaagataccttcaacattactctaactaaggaagaagagaaggttatctctgatttgtgccaaatcatcggtacccgtgctgccagattgtctgtatgctcaattgctggtatctgtagaaaaatgaactacaaatctggacattgtgccgcagatggttcagtttttaatagatacccatactttgaggagcgtgcagctgctgcattgaatgaattattcaagtggaacacagatccaaacgattacccaattaaattgacccatgcagaagatggttctggcgttggtgcggcagtcattgcatgtcttactgaaaaaagactgcttgccggtaagagtgttggtgctaaattatacctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccggcgctagcccccacatacttaagttttcacatattgcgcataataaataggtgcaatctgacccttgccaaataattcgc

SEQ ID NO: 5 - DNA, 4524 bp, synthetic construct
  source 1..4524: mol_type = other DNA
  misc_feature 1..4524: note = synthetic polynucleotide
  misc_difference 536: note = n is a, c, g, or t
aaatgtcgacatagggcacaatagtcagcggggaaacggagttcccgagggtgtcggagatactccagctaattgacccgcgcgccccttaattaactcgagggatcccgggcccctggaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaatggcagtataccctatcaggtattcagatagttttatttttgtaggtatataatctgaagcctttgaactattttctcgtatatatcatggagtatacattgcattagcaacattgcatactagttcataacttcgtataatgtatgctatacgaagttattaattaacaagggcgaattctgcagatatcggccggccgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatgaataatgttttgtgtatcggtgagttgcttattgattttatctgcagtgacatcgacaccaccttgtctaagggagagaacttcaagaagaaagccggtggtgctccagctaacgtcaccgctgccatctctaagttgggtggatctgccagtttccttggtaaggtcggtaacgaccctttcggtcacttcttgaaggagacccttgacgaggtcaaggtcgacacctctatgttgattatggataataattcttctaccaccttggctttcgtctctttgcaagccaacggtgagagggatttcgtctttaatagaggagccgatggtcttttgaggtacgacgaaattaatttggataaagtttatagtaacaaaattatccacttcggatctgccaccgccttgttgggaggagagatgaccgacacctacttgaagatcatggaggaggccaagaagagaggtatcatcatttctttcgatccaaattatagagataatttgtgggaaaatagaactgaagaatttatcgccatcagtagaaagtgcatcgagttggccgacttcgtcaagttgagtgatgaagagcttaaaatcattagtggtgagaaaaatattaaaaatggtgttaagttgttggcctctaacaacaaggttatcgccgtcaccttgggaaaggagggaaccatgatcagtaacggtgaagaagttgagatcatcgaaagtatcaaaatcaaatctatcgactctaccggtgctggtgacgccttcgtcggagccttcttgtacaagttgtctgaggcccttgaagctagagatatcttgagtgatttcaacaaaattaaggaaaacgttagattcgccaacaaggtcggagccatcgtctgcaccaagttgggtgccatctcttctttgccttctcctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccggcgctagcccccacatacttaagttttcacatattgcgcataataaataggtgcaatctgacccttgccaaataattcgc

SEQ ID NO: 6 - DNA, 5219 bp, synthetic construct
  source 1..5219: mol_type = other DNA
  misc_feature 1..5219: note = synthetic polynucleotide
  misc_difference 429: note = n is a, c, g, or t
ccgggcccctggaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaatggcagtataccctatcaggtattcagatagttttatttttgtaggtatataatctgaagcctttgaactattttctcgtatatatcatggagtatacattgcattagcaacattgcatactagttcataacttcgtataatgtatgctatacgaagttattaattaacaagggcgaattctgcagatatcggccggccgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatgtcttctaatcttagtgagaaagctgtcgaccaacagaaggagttcggtttgcttaaatctaccagtagtgtcgattctgtccatgaggtcgaagccggtgtcaaggtcggagaggatgatgataattacttcttttctgaagatcaaattcttacccaaatcaaggtctacgaagacgagatcgccaacactccagttaccaagacccttaggcagaggatgaggaggatggagttctgtgaagtcaacttttctaataaaacctacatggtcatcatgttgggattcttcgcctctttcgccggaatcttgagtggtgtcgatcagtctaccatctctggtgccagttacggtatgaaggcctctttgaaacttacctctgacgaagatagtttgatctcttctcttatgccattgggagccgtcggtggtagtatccttcttacccctttgtctgagtacttcggaaggaaggtcgctttggtcatcagttgtatcttttataccatcggaggtatcttgtgcgccgctgctcaagatgtccacaccatgtacgccggtaggtttcttatcggagtcggtgtcggaatcgagggtggtggagtcggagtttacatcgccgagtctgtcccatctaccgttagaggttctcttgtcagtctttaccagttcaacatcgctcttggtgagttggtcggttacgtcatcggtgtcatcttcttcgacgtcaagggaggatggaggtacatgcttggtagtagtcttgttttttctaccatcttgtttgttggtttgttttttcttccagagtctcctaggtggttgatccacaagggttacgacgtcgaagcctacaaggtttggagaagattgagggacaccagtgaccttggaaacaagagggagttccttgagatgaagcacgctgccgagcaagatagacagcttaaagagcaagagtctagatttaaatctatgtttgacttgatcttgatccctaggaatagaagggcccttctttattctatcatgatggttagtttgggtcagcttaccggtattaacgccatcatgtattatatgtctaccttgatgggacagatcggtttcagtccaaaacaagctgtcgccatgtctatggtcggtggtgctgctcttttgatcggaaccatcccagccatcctttacatggacaagttcggtagaaggccttggagtatgaccatcatcggtttctctgtcggacttgtccttgtcggtgtcggttaccagatcgacttgaacaccaatttggccgctgccgaaggtgtttacttgaccggtcaaatcctttataatatcttcttcggtacctacgctaccttgacttgggtcgtcccttctgagagtttctctttggccactagaagtatcggaatgaccatctgctctgccttcttgtacctttgggccttcaccgttacctataactttaataaaatgaaggacgcttttacctacaccggtttgacccttggtttctacggtggaatcgctattgtcattggtatcccatatcaattgctttttatgccagagaccaaggacaagaccttggaggagatcgatgatatttttgaaatgccaactagacagcttgtcaagcagaatcttaaaaatcttaaagattacgtttctggtaatagaaagagtattcctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccggcgctagcccccacatacttaagttttcacatattgcgcataataaataggtgcaatctgacccttgccaaataattcgcggccgctcgcgacatcacactggcggcgggc

SEQ ID NO: 7 - DNA, 6194 bp, synthetic construct
  source 1..6194: mol_type = other DNA
  misc_feature 1..6194: note = synthetic polynucleotide
  misc_difference 3191: note = n is a, c, g, or t
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgcgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatgaataatgttttgtgtatcggtgagttgcttattgattttatctgcagtgacatcgacaccaccttgtctaagggagagaacttcaagaagaaagccggtggtgctccagctaacgtcaccgctgccatctctaagttgggtggatctgccagtttccttggtaaggtcggtaacgaccctttcggtcacttcttgaaggagacccttgacgaggtcaaggtcgacacctctatgttgattatggataataattcttctaccaccttggctttcgtctctttgcaagccaacggtgagagggatttcgtctttaatagaggagccgatggtcttttgaggtacgacgaaattaatttggataaagtttatagtaacaaaattatccacttcggatctgccaccgccttgttgggaggagagatgaccgacacctacttgaagatcatggaggaggccaagaagagaggtatcatcatttctttcgatccaaattatagagataatttgtgggaaaatagaactgaagaatttatcgccatcagtagaaagtgcatcgagttggccgacttcgtcaagttgagtgatgaagagcttaaaatcattagtggtgagaaaaatattaaaaatggtgttaagttgttggcctctaacaacaaggttatcgccgtcaccttgggaaaggagggaaccatgatcagtaacggtgaagaagttgagatcatcgaaagtatcaaaatcaaatctatcgactctaccggtgctggtgacgccttcgtcggagccttcttgtacaagttgtctgaggcccttgaagctagagatatcttgagtgatttcaacaaaattaaggaaaacgttagattcgccaacaaggtcggagccatcgtctgcaccaagttgggtgccatctcttctttgccttctcctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccggccccatggagatccgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcc

SEQ ID NO: 8 - DNA, 7116 bp, synthetic construct
  source 1..7116: mol_type = other DNA
  misc_feature 1..7116: note = synthetic polynucleotide
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgcgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatgtcttctaatcttagtgagaaagctgtcgaccaacagaaggagttcggtttgcttaaatctaccagtagtgtcgattctgtccatgaggtcgaagccggtgtcaaggtcggagaggatgatgataattacttcttttctgaagatcaaattcttacccaaatcaaggtctacgaagacgagatcgccaacactccagttaccaagacccttaggcagaggatgaggaggatggagttctgtgaagtcaacttttctaataaaacctacatggtcatcatgttgggattcttcgcctctttcgccggaatcttgagtggtgtcgatcagtctaccatctctggtgccagttacggtatgaaggcctctttgaaacttacctctgacgaagatagtttgatctcttctcttatgccattgggagccgtcggtggtagtatccttcttacccctttgtctgagtacttcggaaggaaggtcgctttggtcatcagttgtatcttttataccatcggaggtatcttgtgcgccgctgctcaagatgtccacaccatgtacgccggtaggtttcttatcggagtcggtgtcggaatcgagggtggtggagtcggagtttacatcgccgagtctgtcccatctaccgttagaggttctcttgtcagtctttaccagttcaacatcgctcttggtgagttggtcggttacgtcatcggtgtcatcttcttcgacgtcaagggaggatggaggtacatgcttggtagtagtcttgttttttctaccatcttgtttgttggtttgttttttcttccagagtctcctaggtggttgatccacaagggttacgacgtcgaagcctacaaggtttggagaagattgagggacaccagtgaccttggaaacaagagggagttccttgagatgaagcacgctgccgagcaagatagacagcttaaagagcaagagtctagatttaaatctatgtttgacttgatcttgatccctaggaatagaagggcccttctttattctatcatgatggttagtttgggtcagcttaccggtattaacgccatcatgtattatatgtctaccttgatgggacagatcggtttcagtccaaaacaagctgtcgccatgtctatggtcggtggtgctgctcttttgatcggaaccatcccagccatcctttacatggacaagttcggtagaaggccttggagtatgaccatcatcggtttctctgtcggacttgtccttgtcggtgtcggttaccagatcgacttgaacaccaatttggccgctgccgaaggtgtttacttgaccggtcaaatcctttataatatcttcttcggtacctacgctaccttgacttgggtcgtcccttctgagagtttctctttggccactagaagtatcggaatgaccatctgctctgccttcttgtacctttgggccttcaccgttacctataactttaataaaatgaaggacgcttttacctacaccggtttgacccttggtttctacggtggaatcgctattgtcattggtatcccatatcaattgctttttatgccagagaccaaggacaagaccttggaggagatcgatgatatttttgaaatgccaactagacagcttgtcaagcagaatcttaaaaatcttaaagattacgtttctggtaatagaaagagtattcctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccggccccatggagatccgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgat

SEQ ID NO: 9 - DNA, 7295 bp, synthetic construct
  source 1..7295: mol_type = other DNA
  misc_feature 1..7295: note = synthetic polynucleotide
  misc_feature 4112: note = n is a, c, g, or t
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgcgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtaatggttaagatcgacgctagtgccgaccttgactggaccaatttgcctcctcagcagaggcttggtaggatcatcggaggaaagtacattaacgacagtgttgacgaggctggacctatctctgccgaaagaccattgaccaaggtcgagtgtgaccttcctcttacctacaccgagtctgtcgaaggagacggaagggaatgggttgtcttgactttcgctccagctgacccacaagatccatacaactgggccctttctaggaagtggctttacacctctttgctttgcggtatgaccttgttcatcggttgcgccaccaccgtctatagttctaccatttctaggatggtccttagttttgatagggtcgccgagatcggtcaacttggattgtttaccttcaacatgacttgtgccattgcccctttgttccttgctccattctgcgagcttgtcggtaggtacgttatctacgtcggtgcttatctttgcttctctctttgcttcatcggtcttgcccttggaaggaatatcgccaccatccttgtccttaggaccttgttgggtttgttcggttgtgtcggtaccattcttgttggtggtaccttcgacgacatgttcaagccagaagatagggctatccctatgtctttgtttagttacatcgccattttgggtactgtcggtgcccctatctacgccggatttgttgacgagaacgtcggatggagatggaccgagggtatccaaggtttggccaacatccctttgcttgtcgttatcgttatcttttttagggagactagaggtggagtcacccttaggaagagagcccagttgcttaggaaggataccggtgacgacagatgggtcagtagagaggagttggaagctcccggtttgaaggacgccttgtatgccagtagtgtcaaggccatccagatgttgtgcaccgagccagttgtctttttctttggactttggatcgcctttgcttggttcatcaccttccttttcctttctgtcatctctatcaccttctctgagggtaagcattgggccgaaggtatcgccggtttgccttacatcggtttggttttgggttgcactcttgccttcggtgccaacttcttgcaaatcagaaagtacgattctttgagggccaagtctaacggtcctttgccaccagaaaatagattgtacggagccatgtgcggagccttgttcttgcctattggtctttttatttattctttttctcaataccctagattgcactggatgggacctatggtcggtcttacttgtatctgcttcggaatcttctttatttttgaaagttgttacagttatacctctgactgctacggtcaatctgcctctagtgccatcgccggtcaaggtttcatgagaaacacccttggagccgtctctccattgttcgctcaccagttctttagaaaggtccatagtcagtacgccggtcttatctgctctgtcgtcgccaccggtttgacctttcttccttatatcttgttcaagttcggaccaaccattagagccaagagtaagagagccattgtctacccagctgacaacggtgacattcttccagatgctgagcaacctagtccagacgactctgagcagattagttctccatacgaaaaagaaaggtctcaagaggaaggacacatcgagaacgccaacatcccagaaactaccaccactaccaacgacgagaagaatagacaaggtggagccgaattgcacagttctgacgagagtagtaactctaccgaggacgaggtcgaagacgccttgaggattcagcacgatgatgagcctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccggccccatggagatccgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaa

SEQ ID NO: 10 - DNA, 6963 bp, synthetic construct
  source 1..6963: mol_type = other DNA
  misc_feature 1..6963: note = synthetic polynucleotide
ctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatgcggccgcgcgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacgcacgcgtatggaccttaataatagtgaagactctcagaagttccttccaagtgccacccagttcgagttggagaagcaaagatcttcttctgaaatcttcgaagattctactaatgccaagcttaacaccgagaacggtaaggcctctgacatccttaacatcaatttgaaccttgaggaggagatcgacgcccttaataagtacggtgacagtcttagaaaagaattgtctttttggggtaagaccaaaaggatggagttcgaggttaattttaataatagacaacacatggtctacatgcttggaatcttcgccgccttcgccggtatccttagtggtatcgaccaaagtattatctctggtgcttctattggtatgaaccaaatgttgaacttgaccacccatgaggccagtcttgtcagttctcttatgccacttggtgctgtcgccggatctatcttgcttactcctttctctgaatactttggaagaagaaaatctttggtcattagttgtgttttctacaccatcggagccattatctgcgccgccgccaagaaccagcacgaaatgtatgccggtaggttccttgtcggtgttggtgttggattggagggtggtggtatcggagtttacatcgccgagagtgtcccaagtaccgttagaggttctcttgtcagtctttatcaattcaatattgctttgggtgaattgttcggttatatcatcggagttatcttcttcgacgtcaagggtggatggagatacatggtcggttcttctttgatttttagtactattatgtttatcggtcttttgttcttgccagagtctcctaggtggttgatgcacaagggtagagtcggtgagagttggaacgtctggaagaggttgaggaacattgacatcgaaggtaataaagtcgaattccttgagttgaggcaagttgctgaacaagaaaaggagttgcacaagcacgagtctaggttccaagcttggttcgaccttattaggattcctaggaacagaagagctttggtctatagttctatcatgatctgccttggtcagttgaccggaattaacgccattatgtactatatgagtaccttgatgggaaatattggattttctcctaaaaaatctgtcgctatgtctatggtcggtggagctgccttgttgatcggtaccatcccagccatcctttggatggatagattcggaaggagagcttggagtatgaacttgattatttttgctttcggtcttatcttggttggagttggataccagatcaacatgagaaccaacttgccagccgctgaaggtgtctatttgaccggtcagattatctacaatatggctttcggttcttacgctgctttgacttgggtccttccatctgagtctttcagtttgggtaccagatctgccggaatgaccatcagtagtgccttgttgtacttgtgcgcctttaccgtcacctacaacttcgagaagatgcaagatgccatgacctactctggacttacccttggtttctacggaggtatcgccatcgtcattggtatcccttaccaattgctttgtatgccagaaaccaagaacagaacccttgaggaaatcgatgatattttcgaaaaacctactaggcaaattattagagaaaacctttctcaccttagaaaaggaagaatcagttacctgcaggtttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacaattcgcccttacatatggataacttcgtataatgtatgctatacgaagttatgctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgaaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacaatgtttgctttctactttctcaccgcatgcaccactttgaagggtgttttcggagtttctccgagttacaatggtcttggtctcaccccacagatgggttgggacagctggaatacgtttgcctgcgatgtcagtgaacagctacttctagacactgctgatagaatttctgacttggggctaaaggatatgggttacaagtatgtcatcctagatgactgttggtctagcggcagggattccgacggtttcctcgttgcagacaagcacaaatttcccaacggtatgggccatgttgcagaccacctgcataataacagctttcttttcggtatgtattcgtctgctggtgagtacacctgtgctgggtaccctgggtctctggggcgtgaggaagaagatgctcaattctttgcaaataaccgcgttgactacttgaagtatgataattgttacaataaaggtcaatttggtacaccagacgtttcttaccaccgttacaaggccatgtcagatgctttgaataaaactggtaggcctattttctattctctatgtaactggggtcaggatttgacattttactggggctctggtatcgccaattcttggagaatgagcggagatattactgctgagttcacccgtccagatagcagatgtccctgtgacggtgacgaatatgattgcaagtacgccggtttccattgttctattatgaatattcttaacaaggcagctccaatggggcaaaatgcaggtgttggtggttggaacgatctggacaatctagaggtcggagtcggtaatttgactgacgatgaggaaaaggcccatttctctatgtgggcaatggtaaagtccccacttatcattggtgccgacgtgaatcacttaaaggcatcttcgtactcgatctacagtcaagcctctgtcatcgcaattaatcaagatccaaagggtattccagccacaagagtctggagatattatgtttcagacaccgatgaatatggacaaggtgaaattcaaatgtggagtggtccgcttgacaatggtgaccaagtggttgctttattgaatggaggaagcgtagcaagaccaatgaacacgaccttggaagagattttctttgacagcaatttgggttcaaaggaactgacatcgacttgggatatttacgacttatgggccaacagagttgacaactctacggcgtctgctatccttgaacagaataaggcagccaccggtattctctacaatgctacagagcagtcttataaagacggtttgtctaagaatgatacaagactgtttggccagaaaattggtagtctttctccaaatgctatacttaacacaactgttccagctcatggtatcgccttctataggttgagaccctcggcttaagctcaatgttgagcaaagcaggacgagaaaaaaaaaaataatgattgttaagaagttcatgaaaaaaaaaaggaaaaatactcaaatacttataacagagtgattaaataataaacggcagtataccctatcaggtattgagatagttttatttttgtaggtatataatctgaagcctttgaactattttctcgtatatatcatggagtatacattgcattagcaacattgcatactagttcataacttcgtataatgtatgctatacgaagttattaattaacaagggcgatttctgcagatatcggccggccccatggagatccgcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttagggcc

SEQ ID NO: 11 - DNA, 1831 bp, Kluyveromyces lactis
  source 1..1831: mol_type = other DNA
aaatgttaaagttattgtccttgatggtcccattagcttctgcagctgttatccacagacgtgatgctaacatttcagctattgcatccgaatggaactccacttctaactcttcttcatctttatctttaaacagaccagctgtccattattctccagaggaaggttggatgaacgacccaaacggtttatggtacgatgctaaagaggaagattggcacatctactatcaatactatcctgatgcccctcattggggtttgccattgacttggggtcatgcagtctccaaagatttgaccgtctgggacgaacaaggtgttgcattcggtccagagtttgaaacagcaggtgccttttctggttctatggttattgattacaataacacctccggtttctttaactcatccaccgacccaagacaacgtgtcgttgccatttggactttggattattctggctctgaaacacaacaattatcttattctcatgatggtggttatacattcaccgaatattctgacaaccctgtcttagatattgactcagacgcttttagagatccaaaggttttctggtatcaaggtgaagattccgaatcagaaggtaactgggtcatgacagttgccgaagcagatcgtttctccgtcttaatctactcttctccagaccttaagaattggaccttagaatcaaacttttccagagaaggctacttaggctataactatgagtgtcctggtttagttaaggtcccatacgtcaaaaacaccacatacgcatctgctccaggctcaaatatcacctcatctggtccacttcatccaaattctactgtttctttctcaaattcatcctctattgcatggaatgcttcttccgttccacttaacattactttatccaattctaccttggttgatgaaacttctcaattggaagaagttggttacgcatgggttatgattgtctcattcaatcctggctccattttaggcggttccggtactgaatacttcatcggtgactttaatggtacacacttcgagccacttgataagcaaactagattcttagatttgggtaaagattactacgctttgcaaactttcttcaataccccaaacgaggttgacgttttgggtatcgcatgggcctctaattggcaatatgctaaccaagttccaacagatccatggagatcatccatgtccttggttagaaacttcactatcactgaatacaacatcaattctaatactactgcattggtcttgaactctcaaccagttttagattttacctctttaagaaagaacggcacatcatatactttagagaatcttacattaaactcctcttctcacgaggttttggaatttgaagatcctaccggtgttttcgaattttcccttgaatattccgtcaactttaccggtattcacaactgggtttttaccgacttgtccttgtatttccaaggtgataaggattcagatgaatacttgagacttggttacgaagctaactccaagcagttctttttagatagaggtcattctaacattccatttgttcaagaaaatccattcttcactcagagactttcagtttccaatcctccatcctccaactcctccaccttcgatgtctacggtattgttgacagaaatatcattgaattgtatttcaacaatggtactgttacctctactaacacctttttcttctccactggtaacaatattggttccatcattgttaagtctggtgttgatgacgtctatgaaattgaatcattgaaggttaatcagttttacgttgacta

SEQ ID NO: 12 - protein, 609 aa, Kluyveromyces lactis
MLKLLSLMVPLASAAVIHRRDANISAIASEWNSTSNSSSSLSLNRPAVHYSPEEGWMNDPNGLWYDAKEEDWHIYYQYYPDAPHWGLPLTWGHAVSKDLTVWDEQGVAFGPEFETAGAFSGSMVIDYNNTSGFFNSSTDPRQRVVAIWTLDYSGSETQQLSYSHDGGYTFTEYSDNPVLDIDSDAFRDPKVFWYQGEDSESEGNWVMTVAEADRFSVLIYSSPDLKNWTLESNFSREGYLGYNYECPGLVKVPYVKNTTYASAPGSNITSSGPLHPNSTVSFSNSSSIAWNASSVPLNITLSNSTLVDETSQLEEVGYAWVMIVSFNPGSILGGSGTEYFIGDFNGTHFEPLDKQTRFLDLGKDYYALQTFFNTPNEVDVLGIAWASNWQYANQVPTDPWRSSMSLVRNFTITEYNINSNTTALVLNSQPVLDFTSLRKNGTSYTLENLTLNSSSHEVLEFEDPTGVFEFSLEYSVNFTGIHNWVFTDLSLYFQGDKDSDEYLRLGYEANSKQFFLDRGHSNIPFVQENPFFTQRLSVSNPPSSNSSTFDVYGIVDRNIIELYFNNGTVTSTNTFFFSTGNNIGSIIVKSGVDDVYEIESLKVNQFYVD

SEQ ID NO: 13 - DNA, 719 bp, Issatchenkia orientalis
  source 1..719: mol_type = genomic DNA
cgtccaggagtccatcggttcctgtcagatgggatactcttgacgtggaaaattcaaacagaaaaaaaacccccaataatgaaaaataatactacgttatatccgtggtatcctctatcgtatcgtatcgtagcgtatcgtaccgtaccgtatcacagtatagtctaatattccgtatcttattgtatcctatcctattcgatcctattgtatttctgtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggttagcgtatcccccttcctcggcttttacaaatgatgcgctcttaatagtgtgtcgttatccttttggcattgacgggggagggaaattgattgagcgcatccatattttggcggactgctgaggacaatggtggtttttccgggtggcgtgggctacaaatgatacgatggtttttttcttttcggagaaggcgtataaaaaggacacggagaacccatttattctaataacagttgagcttctttaattatttgttaatataatattctattattatatattttcttcccaataaaacaaaataaaacaaaacacagcaaaacacaaaaattctagacg

SEQ ID NO: 14 - DNA, 921 bp, Issatchenkia orientalis
  source 1..921: mol_type = genomic DNA
tttggatgaggcattcaatgtctccatgtttgaagaccagagaaagtgtaggtaacacaatagccatggacaattagcgaatccgaatatgtgtactaacattgtgcctccaactgacagctggtcgaataatgattgaaatcatcaaacatatcagagatatatcccagaaccatttaacaactgttattgtatgtatgcattactgttttacattgtgtctttccaaatactaacattgccattatccaatccatacatatatatatatatagattacacaacgcaaacctcgacagtcgattggtttacatcgtgtaagcagccttttacggaatatgatggacaaccagatcctcatagatggagacaatgtgcagatagattccatttatgatgacattgatgtgtttttaaatgcaaaataatctcctatataagtgagttccgagtctccccatgcttatcttatcgtgttttttctgtaaattaggtcaaagtattcaaacaattccctcaatctttcccattttttgctgagcccccatatcatcaaattaacgtatcaaatttcctaattagtagccccgtatgttaaattagacatgtgtgactgccatcccggacagcctacccaatgacacccgcgcaccgcacatattgtgtttagtgcgcgccgtctgctgaagcgactccctgtttgggaggaaccgagggcgggttgcccgatcccttgcccctcgctcctcctcctgggctcccccttgcagagggacaccgaggggatccctcgtgtgagagcttggaggtggatggtggtcaattttctcatttgattgaaagatttgttatattgaaaattcagtttgtggaagttgtgattaaaaggttttactgtttgttctgtagacacattcaata

SEQ ID NO: 15 - DNA, 624 bp, Issatchenkia orientalis
  source 1..624: mol_type = other DNA
  misc_feature 363: note = n is a, c, g, or t
ctgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaaca

SEQ ID NO: 16 - DNA, 453 bp, Issatchenkia orientalis
  source 1..453: mol_type = genomic DNA
attgcagccaccatcaatttaacattggttttcaaataatcagttaaacctgacagtgattaccagccacggggattcgagaaattcgagcaattcgaaagagccgcaaaaaaataagcaagaaaaaagaaaataaaaaacaaaaaaaacatgaaaaaaacatgaaaaaaacatgaaaaaaaaaaaaaagaaagaaaaaaaaaatcaattttttcatcagaggcattggtaaaacactctgtgctgtcgcgacacgacgaacgggatgccatggtacttgtttcgggtgtctctcctcctttggaaactctttccgctctcgaaaaaattaataccgtagaggaggggacatgaatagaatcgtatataacagggtcattccctgatgctgtctttgtaaggagccatttattgtcatttgtttataccaaccccattgaacaaacaagaaaa

SEQ ID NO: 17 - DNA, 471 bp, Issatchenkia orientalis
  source 1..471: mol_type = genomic DNA
taacctgatccaaaaggggtatgtctattttttagagtgtgtctttgtgtcaaattatggtagaatgtgtaaagtagtataaactttcctctcaaatgacgaggtttaaaacaccccccgggtgagccgagccgagaatggggcaattgttcaatgtgaaatagaagtatcgagtgagaaacttgggtgttggccagccaagggggaaggaaaatggcgcgaatgctcaggtgagattgttttggaattgggtgaagcgaggaaatgagcgacccggaggttgtgactttagtggcggaggaggacggaggaaaagccaagagggaagtgtatataaggggagcaatttgccaccaggatagaattggatgagttataattctactgtatttattgtataatttatttctccttttatatcaaacacattacaaaacacacaaaacacacaaacaaacacaattacaaaaa

SEQ ID NO: 18 - DNA, 535 bp, Issatchenkia orientalis
  source 1..535: mol_type = genomic DNA
gctctacaagcctcatatgggttcaatgggtctgcaatgaccgcatacggacttggacaattaccttctattgaatttctgagaagagatacatctgaccagcaatgtaagcagacaatcccaattctgtaaacaacctctttgtccataattccccatcagaagagtgaaaaatgccctcaaaacgcatgcgccactcccacctctcagctgcactgcgccacctctgagggtcctttcaggggtcgactaccccggacacctcgcagaggagcgacgtcacgtacttttaaaatggcagagacgcgcagtttcttgaagaaaggataaaaatgaaatggtgcggaaatgcgaaaatgatgaaaaattttcttggtggcgaggaaattgagtgcaataattggcacgaggttgttgccacccgagtgtgagtatatatcctagtttctgcacttttcttcttcttttccttgcgttttcttttcaactttttttactttttccttcaacagacaaatctaacttatatatcaca

SEQ ID NO: 19 - DNA, 991 bp, Issatchenkia orientalis
  source 1..991: mol_type = genomic DNA
ttctttccccctctcaagctggcgtgaaatgcaaccttacggcgtctacgttactacaaggtccagaaagtgtaggtattgctactatttttattttttattggttctggagaaatgcagacagtcaatgaacacaactgtctcaatatgcatctatgcacatgcacacacacacacatcacaggtacccctacaaagagaggtctcttgataatgtttcattaccacgtggcatcccccccccccccccaataaacaagtggccgagttcccctgttgcagaggaggacaaaaaaaccgctggtgttggtaccattatgcagcaactagcacaacaaacaaccgacccagacatacaaatcaacaacacttcgccaaagacaccctttccagggaggatccactcccaacgtctctccataatgtctctgttggcccatgtctctgtcgttgacaccgtaaccacaccaaccaacccgtccattgtactgggatggtcgtccatagacacctctccaacggggaacacctcattcgtaaaccgccaaggttaccgttcctcctgactcgccccgttgttgatgctgcgcacctgtggttgcccaacatggttgtatatcgtgtaaccacaccaacacatgtgcagcacatgtgtttaaaagagtgtcatggaggtggatcatgatggaagtggactttaccacttgggaactgtctccactcccgggaagaaaagacccggcgtatcacgcggttgcctcaatggggcaatttggaaggagaaatatagggaaaatcacgtcgctctcggacggggaagagttccagactatgaggggggggggtggtatataaagacaggagatgtccacccccagagagaggaagaagttggaactttagaagagagagataactttccccagtgtccatcaatacacaaccaaacacaaactctatatttacacatataaccccctccaaccaaaca

SEQ ID NO: 20 - DNA, 284 bp, Issatchenkia orientalis
  source 1..284: mol_type = genomic DNA
taacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaa

SEQ ID NO: 21 - DNA, 256 bp, Saccharomyces cerevisiae
  source 1..256: mol_type = other DNA
tttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccg

SEQ ID NO: 22 - DNA, 240 bp, Saccharomyces cerevisiae
  source 1..240: mol_type = other DNA
gctcaatgttgagcaaagcaggacgagaaaaaaaaaaataatgattgttaagaagttcatgaaaaaaaaaaggaaaaatactcaaatacttataacagagtgattaaataataaatggcagtataccctatcaggtattcagatagttttatttttgtaggtatataatctgaagcctttgaactattttctcgtatatatcatggagtatacattgcattagcaacattgcatactagt

SEQ ID NO: 23 - DNA, 9630 bp, synthetic construct
  source 1..9630: mol_type = other DNA
  misc_feature 1..9630: note = synthetic plasmid
  misc_feature 3086: note = n is a, c, g, or t
  misc_feature 5897: note = n is a, c, g, or t
  misc_feature 6283: note = n is a, c, g, or t
  misc_feature 6314: note = n is a, c, g, or t
  misc_feature 6343: note = n is a, c, g, or t
  misc_feature 6347: note = n is a, c, g, or t
  misc_feature 6371: note = n is a, c, g, or t
  misc_feature 6525: note = n is a, c, g, or t
  misc_feature 6543: note = n is a, c, g, or t
  misc_feature 7386: note = n is a, c, g, or t
  misc_feature 7397: note = n is a, c, g, or t
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggtcgaggagtccatcggttcctgtcagatgggatactcttgacgtggaaaattcaaacagaaaaaaaaccccaataatgaaaaataacactacgttatatccgtggtatcctctatcgtatcgtatcgtagcgtatcgtagcgtaccgtatcacagtatagtctaatattccgtatcttattgtatcctatcctattcgatcctattgtatttcagtgcaccattttaatttctattgctataatgtccttattagttgccactgtgaggtgaccaatggacgagggcgagccgttcagaagccgcgaagggtgttcttcccatgaatttcttaaggagggcggctcagctccgagagtgaggcgagacgtctcggtcagcgtatcccccttcctcggcttttacaaatgatgcgctcttaatagtgtgtcgttatccttttggcattgacgggggagggaaattgattgagcgcatccatatttttgcggactgctgaggacaatggtggtttttccgggtggcgtgggctacaaatgatacgatggtttttttcttttcggagaaggcgtataaaaaggacacggagaacccatttattctaaaaacagttgagcttctttaattattttttgatataatattctattattatatattttcttcccaataaaacaaaataaaacaaaacacagcaaaacacaaaaaggatccatgtctaatttacttactgttcaccaaaacttgcctgcattaccagttgacgcaacctccgatgaagtcagaaagaaccttatggatatgtttagagatagacaagctttctccgaacatacttggaaaatgttattatccgtttgtagatcctgggccgcttggtgtaaacttaacaatagaaaatggtttcctgctgaaccagaagacgtcagagattacttactttacttacaagctagaggtttggctgttaaaactatccaacaacacttaggtcaattgaatatgttacacagaagatccggtttaccaagaccatccgattccaacgcagtttcccttgttatgagaagaattagaaaagaaaatgttgacgctggtgaaagagctaaacaagcattagcatttgaaagaaccgatttcgatcaagttagatccttaatggaaaattccgatagatgtcaagatattagaaacttagctttcttaggtattgcttacaacacattattaagaatcgctgaaattgctagaattagagttaaagatatttcaagaaccgatggcggtagaatgttaatccacattggcagaacaaaaaccttagtctccacagcaggcgtcgaaaaagcattatcattaggtgttactaaattagttgaacgttggatttccgtttccggtgttgcagatgacccaaacaactacttattctgtcgtgttagaaaaaatggtgttgccgctccttccgctacctcacaattatccacaagagcattagaaggcatttttgaagctacccacagacttatttatggtgcaaaagacgattccggtcaaagatatttagcttggtctggtcattccgctagagttggtgccgcaagagacatggcaagagctggtgtttctattcctgaaattatgcaagccggtggttggactaatgttaacattgttatgaactatatcagaaacttagattccgaaacaggtgctatggttagattacttgaagacggtgattaagttaattaacatctgaatgtaaaatgaacattaaaatgaattactaaactttacgtctactttacaatctataaactttgtttaatcatataacgaaatacactaatacacaatcctgtacgtatgtaatacttttatccatcaaggattgagaaaaaaaagtaatgattccctgggccattaaaacttagacccccaagcttggataggtcactctctattttcgtttctcccttccctgatagaagggtgatatgtaattaagaataatatataattttataataaaagaattcggcagatctggatcgatcccccgggctgcatgcaacggcaacatcaatgtccacgtttacacacctacatttatatctatatttatatttatatttatttatttatgctacttagcttctatagttagttaatgcactcacgatattcaaaattgacacccttcaactactccctactattgtctactactgtctactactcctctttactatagctgctcccaataggctccaccaataggctctgtcaatacattttgcgccgccacctttcaggttgtgtcactcctgaaggaccatattgggtaatcgtgcaatttctggaagagagtgccgcgagaagtgaggcccccactgtaaatcctcgagggggcatggagtatggggcatgnaggatggaggatggggggggggggggaaaataggtagcgaaaggacccgctatcaccccacccggagaactcgttgccgggaagtcatatttcgacactccggggagtctataaaaggcgggttttgtcttttgccagttgatgttgctgagaggacttgtttgccgtttcttccgatttaacagtatagaatcaaccactgttaattatacacgttatactaacacaacaaaaacaaaaacaacgacaacaacaacaacctgcaggaaatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacactagtgggtttttcaatgatactattgatccaagacaaagatgcgttgcaatttggacttataacactcctgaaagtgaagagcaatacattagctattcccttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctcggatgacttgaagtcctggaagttagaatctgcatttgccaatgaaggtttcttaggctaccaatatgaatgtccaggtttgattgaagtcccaactgagcaagatccttccaaatcctattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttcgttgaacactgaatatcaagctaatccagagactgaattgatcaatttgaaagccgaaccaatattgaacattagtaatgctggcccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggcaacgctctaggatctgtgaacatgaccactggtgtcgataatttgttctacattgacaagttccaagtaagggaagtaaaatagcctgcaggcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgaattcccgggccttaccgtcgacgaatttcagcattttcatttcaaggcgatattatgtttcactaaactcaggacaggaatatactaagaataactacaacatacacacaacataagccaagatggatcaacttaactaccaagaacaacaacaatttcaaaagatcgttgaacaaaagcaaatggctgatttcatgaggctatgaattcgcccttgatctgggtgtatactgcacaacctcattgttcgggaatttgattctcatctcacatacaggcctgtagtattgcgccctctccttctccttctccttctccttctccaagagagacttctctctcatcgccctcgtcatcaatggctgctcgctgtattgtcgttggagcatctcccgatacttctgcaactgtgataaactcatctcaggtgacccatccgattctgtatcggtgtctccatctggggctacatctcgggccagtctagatttaaactttgcagaaccttcactttgggggatatacactagtgtctctcccgtgactacatcaccgacaccctcaactgtaccattattattgtcattgttttcctctaagttctcgctttggtcntcatccatctctccttcgggtgctgtatcactcttgatgatttctctaaccctaatacggagactgtgattgcctgaaataatacccacatctttcaacttctgatgaagtgaatctccagagatgaccttcatcagcacttgcacatcaaccacatcaccctccttttgagcatccctcatgattccatagactacatcccgtagcgtctccttgttcttgtacttcttaacaacagtctcgccacagacatggcccctgataatcacctcctgtctctcctcatggccatcctggtcgccattgtcttcgtcgctcggctcaattgccaatgtagcaccctgtggaagattgcttagtctgtatggaacagactcatcaactcntttgccattatgcattaacttgtactttcgnccttggctaagttgaaaatgtttacatcnwtcntcaagtacattggacatgattgtncctgcattgacatttgtccggtaagtcctaaacccactcgctagattcactgtaggcatattcaatcacgttccgtttgaaaaaaaggaaaccaatttattatctccagaaatagttggcgtcttgcatcttgtttggtcttgatctttcgtgnttttttttttttctgtcnttttttttctcctctctccaactttttgatttttagtgtaccaaatcgcactgcttatccacattcatcataaagrgggggggagaagaggggcagaaaataaaaggccatgtcacgtgcctgtgcatttatttgtgtgtgtgtcacgtgctcaaaatgtcttttttttacgtttttaacattttccctttctgtagttgaatccatttgcatgagtcgtacatratgtttgctgtatttacgttaagacactaattcaaatgacaaacagctattattcttagccattaatgcatttttgcaaatctttaactggatttaactatggctaggtraatttgttctggacatcattgccttgacttgttttagtgccgatgtccttatcacttacactcgtaacacaacacaacagcagctaatgttgttgtgtatcgcttgacccttaataactgattcttttttgatgaatgttaagaagaaacaaacaaraaaataaaatcaaaacaggcttcttttgacctctttcaagagaaggttttcttggttgtttcatataccaagatctgaatatcttctattattatacaaaccactgattatacaaatctattcatcgacagtatgarctacgaaaacacactgataaaragagtcatttcttccccttctttttctttttctttttcttcttcttcttagtatccccatcttcattaactccaccaagtagatcctctacaccccccatggccgttaaaaaatgttcacgaaagaaatccatatcattattcttaccatccattaaactgtttagatagatggtgatcatctcccttgcantgtctatatcntcaacgtcgagtaaatgcgacgcaatggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 24 - protein, 463 aa, Issatchenkia orientalis
MTQTLQAAFEKTKSELTVTTEKLEEITNHFVEELEKGISPAGGNIPMNPTWVMDYPLGSETGEYLAIDLGGTNLRVIRVTLQGDSKFKSVNEKYPIPVPMRTGNKDDLFDFIAKSLDDFIKHQYGEDYKGEKLPLGFTFSYPAHQNAINHGVLQTWTKGFDIPGVEGHDVVPMLQASLDKLNTPVEVVALINDTTGTLVASKYCDTETIMGLIFGTGCNGAYYDFAKNIPKLEGKLASDINDETPMAINCEYGAFDNDWKVLPRTKYDIQIDQESPRPGQQFYEKMIAGYYLGEVLRLILLDYYSQGLVFINQDIKSLQVPFAMDTSFPSMIEEHGPEYAGRLFKDTFNITLTKEEEKVISDLCQIIGTRAARLSVCSIAGICRKMNYKSGHCAADGSVFNRYPYFEERAAAALNELFKWNTDPNDYPIKLTHAEDGSGVGAAVIACLTEKRLLAGKSVGAKL

SEQ ID NO: 25 - protein, 477 aa, Issatchenkia orientalis
MTNSKLDLYTEKLHKDFHVSTVSLHKIIDYFIEELDRGLQDGTDPLGIPMNVAWVMEYPTGDETGDYLALDMGGTNLRVVIVHLKGKGQMETDQAFYKLPDGIRTVKDRNVLFDFMAEKLDQFLKDKHPEGIPENKVFPLGFTFSYPATQSRINSGVLQRWTKGFDIPGVEGEDVVPLLTEKLKERNVPIKIVALINDTCGTLVASRYVDPLTEMGCIFGTGVNGAYYEKVSNITKYKGRFPKDIPEDSPMLINCEYGSFDNAHKVIPRTRFDITIDETSPRPGQQTFEKMTAGFYLGDLLRLTLLEAYTEGITFEDAPAEFVKCLNTPQCIDASFLSRIELDESDDLVTAKKLFADEFKYPNATTDEVKFAKAAAQLISNRAARLSICGVAAVCKKRGYKKCHIAADGSVYLKYPNFASRAAEGLADVFGWGHEMPQSEHPIRIVTALDGSGVGAAIIAALAHKREVAGLSLGLED

SEQ ID NO: 26 - protein, 315 aa, Clostridium acetobutylicum
MNNVLCIGELLIDFICSDIDTTLSKGENFKKKAGGAPANVTAAISKLGGSASFLGKVGNDPFGHFLKETLDEVKVDTSMLIMDNNSSTTLAFVSLQANGERDFVFNRGADGLLRYDEINLDKVYSNKIIHFGSATALLGGEMTDTYLKIMEEAKKRGIIISFDPNYRDNLWENRTEEFIAISRKCIELADFVKLSDEELKIISGEKNIKNGVKLLASNNKVIAVTLGKEGTMISNGEEVEIIESIKIKSIDSTGAGDAFVGAFLYKLSEALEARDILSDFNKIKENVRFANKVGAIVCTKLGAISSLPSSEVEGD

SEQ ID NO: 27 - protein, 566 aa, Kluyveromyces lactis
MSSNLSEKAVDQQKEFGLLKSTSSVDSVHEVEAGVKVGEDDDNYFFSEDQILTQIKVYEDEIANTPVTKTLRQRMRRMEFCEVNFSNKTYMVIMLGFFASFAGILSGVDQSTISGASYGMKASLKLTSDEDSLISSLMPLGAVGGSILLTPLSEYFGRKVALVISCIFYTIGGILCAAAQDVHTMYAGRFLIGVGVGIEGGGVGVYIAESVPSTVRGSLVSLYQFNIALGELVGYVIGVIFFDVKGGWRYMLGSSLVFSTILFVGLFFLPESPRWLIHKGYDVEAYKVWRRLRDTSDLGNKREFLEMKHAAEQDRQLKEQESRFKSMFDLILIPRNRRALLYSIMMVSLGQLTGINAIMYYMSTLMGQIGFSPKQAVAMSMVGGAALLIGTIPAILYMDKFGRRPWSMTIIGFSVGLVLVGVGYQIDLNTNLAAAEGVYLTGQILYNIFFGTYATLTWVVPSESFSLATRSIGMTICSAFLYLWAFTVTYNFNKMKDAFTYTGLTLGFYGGIAIVIGIPYQLLFMPETKDKTLEEIDDIFEMPTRQLVKQNLKNLKDYVSGNRKSI

SEQ ID NO: 28 - protein, 616 aa, Zygosaccharomyces bailii
MVKIDASADLDWTNLPPQQRLGRIIGGKYINDSVDEAGPISAERPLTKVECDLPLTYTESVEGDGREWVVLTFAPADPQDPYNWALSRKWLYTSLLCGMTLFIGCATTVYSSTISRMVLSFDRVAEIGQLGLFTFNMTCAIAPLFLAPFCELVGRYVIYVGAYLCFSLCFIGLALGRNIATILVLRTLLGLFGCVGTILVGGTFDDMFKPEDRAIPMSLFSYIAILGTVGAPIYAGFVDENVGWRWTEGIQGLANIPLLVVIVIFFRETRGGVTLRKRAQLLRKDTGDDRWVSREELEAPGLKDALYASSVKAIQMLCTEPVVFFFGLWIAFAWFITFLFLSVISITFSEGKHWAEGIAGLPYIGLVLGCTLAFGANFLQIRKYDSLRAKSNGPLPPENRLYGAMCGALFLPIGLFIYSFSQYPRLHWMGPMVGLTCICFGIFFIFESCYSYTSDCYGQSASSAIAGQGFMRNTLGAVSPLFAHQFFRKVHSQYAGLICSVVATGLTFLPYILFKFGPTIRAKSKRAIVYPADNGDILPDAEQPSPDDSEQISSPYEKERSQEEGHIENANIPETTTTTNDEKNRQGGAELHSSDESSNSTEDEVEDALRIQHDDE

SEQ ID NO: 29 - protein, 566 aa, Saccharomyces carlsbergensis
MDLNNSEDSQKFLPSATQFELEKQRSSSEIFEDSTNAKLNTENGKASDILNINLNLEEEIDALNKYGDSLRKELSFWGKTKRMEFEVNFNNRQHMVYMLGIFAAFAGILSGIDQSIISGASIGMNQMLNLTTHEASLVSSLMPLGAVAGSILLTPFSEYFGRRKSLVISCVFYTIGAIICAAAKNQHEMYAGRFLVGVGVGLEGGGIGVYIAESVPSTVRGSLVSLYQFNIALGELFGYIIGVIFFDVKGGWRYMVGSSLIFSTIMFIGLLFLPESPRWLMHKGRVGESWNVWKRLRNIDIEGNKVEFLELRQVAEQEKELHKHESRFQAWFDLIRIPRNRRALVYSSIMICLGQLTGINAIMYYMSTLMGNIGFSPKKSVAMSMVGGAALLIGTIPAILWMDRFGRRAWSMNLIIFAFGLILVGVGYQINMRTNLPAAEGVYLTGQIIYNMAFGSYAALTWVLPSESFSLGTRSAGMTISSALLYLCAFTVTYNFEKMQDAMTYSGLTLGFYGGIAIVIGIPYQLLCMPETKNRTLEEIDDIFEKPTRQIIRENLSHLRKGRISY

SEQ ID NO: 30 - protein, 323 aa, Lactobacillus helveticus
MAREEKPRKVILVGDGAVGSTFAFSMVQQGIAEELGIIDIAKERVEGDAIDLADATPWTSPKNIYAADYPDCKDADLVVITAGAPQKPGETRLDLVNKNLKILSSIVEPVVESGFEGIFLVVANPVDILTHATWRMSGFPKDRVIGSGTSLDTGRLQKVIGKMENVDPSSVNAYMLGEHGDTEFPAWSYNNVGGVKVADWVKAHNMPESKLEDIHQEVKDMAYDIINKKGATFYGIGTASAMIAKAILNDEHRVLPLSVPMDGEYGLHDLHIGTPAVVGRKGLEQVIEMPLSDKEQELMTASADQLKKVMDKAFKETGVKVRQ

SEQ ID NO: 31 - protein, 323 aa, Lactobacillus helveticus
MAREEKPRKVILVGDGAVGSTFAFSMVQQGIAEELGIIDIAKEHVEGDAIDLADATPWTSPKNIYAADYPDCKDADLVVITAGAPQKPGETRLDLVNKNLKILSSIVEPVVESGFEGIFLVVANPVDILTHATWRMSGFPKDRVIGSGTSLDTGRLQKVIGKMENVDPSSVNAYMLGEHGDTEFPAWSYNNVAGVKVADWVKAHNMPESKLEDIHQEVKDMAYDIINKKGATFYGIGTASAMIAKAILNDEHRVLPLSVPMDGEYGLHDLHIGTPAVVGRKGLEQVIEMPLSDKEQELMTASADQLKKVMDKAFKETGVKVRQ

SEQ ID NO: 32 - protein, 575 aa, Issatchenkia orientalis
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 33 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 34 - protein, 581 aa, Schizosaccharomyces pombe
MFLKYILASGICLVSLLSSTNAAPRHLYVKRYPVIYNASNITEVSNSTTVPPPPFVNTTAPNGTCLGNYNEYLPSGYYNATDRPKIHFTPSSGFMNDPNGLVYTGGVYHMFFQYSPKTLTAGEVHWGHTVSKDLIHWENYPIAIYPDEHENGVLSLPFSGSAVVDVHNSSGLFSNDTIPEERIVLIYTDHWTGVAERQAIAYTTDGGYTFKKYSGNPVLDINSLQFRDPKVIWDFDANRWVMIVAMSQNYGIAFYSSYDLIHWTELSVFSTSGYLGLQYECPGMARVPVEGTDEYKWVLFISINPGAPLGGSVVQYFVGDWNGTNFVPDDGQTRFVDLGKDFYASALYHSSSANADVIGVGWASNWQYTNQAPTQVFRSAMTVARKFTLRDVPQNPMTNLTSLIQTPLNVSLLRDETLFTAPVINSSSSLSGSPITLPSNTAFEFNVTLSINYTEGCTTGYCLGRIIIDSDDPYRLQSISVDVDFAASTLVINRAKAQMGWFNSLFTPSFANDIYIYGNVTLYGIVDNGLLELYVNNGEKTYTNDFFFLQGATPGQISFAAFQGVSFNNVTVTPLKTIWNC

SEQ ID NO: 35 - protein, 627 aa, Aspergillus niger
MHILPGSQHAAELDNSGTLIHSVHCDPEQKAKNIPQSTGIAQASSEWRPSYHLAAPRGWMNDPCGLGYDPTTGLYHLSFQWNPHGNDWGNISWGHATSSDLVSWQISPEPCLTPSAEYDRCGVFTGCFRSHGPDGKPGVLTYVYTSVNHLPLHYTLPYVKGSESLSIAVSRDHGKTWQRIDSNPIHPGAPAGLEVTGWRDPYLNCWPSLRAQRQGGVASPDLYGFISGGIAKESPTVFVYVVNPDNLTEWTYIGPLLHVGLNYRPSRWSGDLGVNWEVANFFTLTDGGVSRDIVIFGAEGCLSCEVGSKRVPRSLLWMCINVRPGLQAQSSGEPLADYSFSGIFDHGCCYAANSFWDPVTEEYVVYCWITEEDLPDRLRHRQGWSGIMSLPRLVRLVTLHNVKRAHQSKLESITSVEIERHSQGTQVRTLSVRPDPRLNILRTSARELHLSNVQLGSVAHQPPAFLPLRTARWEMTATFVIGTHCAAVGLEIGHSPDFHQRTTLSWIPYDETFTIERPPLHDAGINHVPETAPHTLFTFCNNEGEEVTEPLQIHAYFDASVLEVFVNSRTVISTRIYTPHAQVCTGLKFFASATESQPKPSTSAPAAVLVRADIWDGLSVIRDEIKH

SEQ ID NO: 36 - protein, 615 aa, Botryotinia fuckeliana
MSDFPTKEVVNHEDVSSSHYDTQAKMSEEDVSDVGNEKGAAQARPSHIGRKVSEWEALQIAAAGDMETIDNEVQDLEAEVTSRGKSRSDIFQLSFKDPRHYTWLLVAFASMGGLLSGIDQSLISGANLYMPSSLGLNTKQVSLVSSGVPLGAVGGALMLGPLNEAVGRKQAIVWSLFLYTVGAALEAGAVNFGMMMAGRVILGLGVGLEGGTVPVYVAESVAPKYRGNLVSLYQFMIALGEVLGYVVAAIFVGVKSGGWRYMLGSSLVFSTIMLVGILFMPESPRFLMHKGRTADAWGVWKRIRGTDEAARKEFFVMKHSVDSEREMEGARKRFAWLDFITVPRARRAIVYANVMIFLGQFTGINAIQYYMATLMQQVGFNDKEAVFMSLVGGGALLIGTIPAILYMEKFGRRFWAIAMLPGFFIGLVIIGCSYLIPLTNKAGSQGTYITGLILYEGFFGSYACLTWVIPSEVYPTYLRSYGMETSDTMLFLCSFLVTYFFAEMQKAMSNIGLTLGFYGGIAVVGWFYQVLFMPETKNKTLEEIDVIFSQPTGQLVKENLRSSMVTASDFIHGRWGKVFSPVSESDIFRKENGHKSDEGGMGDAKHEELMQPESM

SEQ ID NO: 37 - protein, 1157 aa, Ganoderma boninense
MVVPTMAFGWIVVAVRKVLSSPSPPAVIGALFPPPLNFVSALAIVACLSSADTVISAAIVTASSRRTFSAGFTIDWDWDGLTAACCTSLARTAFIAASEARHTPFDRPQLRFTGTTRERVASQAVAGGHQAPSCASAPLSIIRYLISLDWHTISADEALQRLRVSPQTGLDNAQVQRRQQESGRNVITQPRRNVLRKLLEWVLGGFGSLLLVASILCFVSCTRSNRKPLGEPHPQISNLALGVVLLVVLFLQGAFNAWQDFSTSKVMSSIKDMLPSDVVILRDGNKVKLPAAELVNGDLVYIGLGEKVPADLRLIQVSLDFRFDRSIITGESEPIAGRVDPTSQNFLEVKPRLQYLHTMNTDRAPQTKNIALQGTHCVGGSGVGLVIQTGDNTVFGRIAKLSSMESSGMTTLQREVLFLVMLIASLATAIAVLLVVLWATWLRHSYPSYINVSGLLIDIVSVMVAFLPDGLPLAVTLSIAKIAHTLSQHKVLCKSLVIVETLGAVNVLCSDKTGTLTQNKMHVVNVAVNDVDYESETFGKILADATPEAAANLSQVVAVGGLCNSAIIGGGKEGGSGRSIMGNETDVAVLRFSEAVMSVDSLRDDWIEIFRVNFNSKNKYMLKLFKCADTNAPAPVAAWDNFAPSDSLLVVKGAPDVLLPRCKFALDPQGGKPIPLSDSAMERITRVQENWARDGQRVLLLARRVVREDEIPKSADPLSEEFGELVETLRNDLIIVGMVGLIDPLKPSIPHVVKTCRRAGIRFFIVTGDHPTTSVAIAARAGIVTNAERIHRCADLSTAVADSDRATLDAETPQGIVITGPELDELDAAQVERLCGYEEIVFARTTPEQKLRIVNEFKRRACVVAMTGDGVNDAPSLKAADCGIAMGDGSDVAREAADMVLLDDFSAIIVGLKYGRLVFDNLKKTVLYLIPAGSFSELVPILSNVLIGVPQLLSNIQMILICVATDVLPALSLCLEPPEMDLLERPPRNVKTEHLVNWRLMLHAYGFLGILESLCAMSMSFWYLQRNGVPFSNLILGFGNWPGLTDELLFRAQSVNFFTLILMQWGNLFATRGRKLSIVQHRPTSNPWVFAGAGAALSIGIFFHYVPWFHGIFQTRPVPVEFFFIPLTFALGLLLLDEVRKLVVRRYPKSLLARIAW

SEQ ID NO: 38 - DNA, 70 bp, Issatchenkia orientalis
  source 1..70: mol_type = genomic DNA
attcgccacattcgacacattcgctgcattcgccacaacgtcacgtgaggcatcgtagttttgagttttt

SEQ ID NO: 39 - DNA, 70 bp, Issatchenkia orientalis
  source 1..70: mol_type = genomic DNA
cgtgaacagcttgaacaatttcctgatctcacagcacatttctacgacaaaaaaagaaaacaaacattaa

SEQ ID NO: 40 - DNA, 70 bp, Issatchenkia orientalis
  source 1..70: mol_type = genomic DNA
gacatagggcacaatagtcagcggggaaacggagttcccgagggtgtcggagatactccagctaattgac

SEQ ID NO: 41 - DNA, 70 bp, Issatchenkia orientalis
  source 1..70: mol_type = genomic DNA
ccccacatacttaagttttcacatattgcgcataataaataggtgcaatctgacccttgccaaataattc

SEQ ID NO: 42 - DNA, 865 bp, Issatchenkia orientalis
  source 1..865: mol_type = genomic DNA
ggtaccgagctctaagtagtggtgttggtgaactcaagatggactctttaggtaattatattcttgaatagttgtgtaaagcgaatatgcaaatagatttgttttataattatgcatctctttgaaagaggtttagaggcaaagttcttgcatacaatattgtgattgttttaatgtcattcttgattttcataaagagattaaaaaaaaaaaaaaaaaacttataaaattgagtagaaccatttatatataagacaaagattgtctgtattagtcctcaacacactaaaccttacatacttagggtaaatttgctaatagagtgatatgttcatgagaactccaacgacaacacaaccacctatttgcacaacaaacaccattgtcgcacgctgcgcgccctagaagtagaaagaaagggaaatgacattaagagaatcataccccgtgcccgtaacgccgaaaaaatcacaccccgtcccccacaccttaaaacctcaaccgcttaacaccgccacaccctttctctttataaacgccgtttgcattactcattcttcttataaaccgcaccccccaaaacgcggaatagcttcaaccccccaatcagatatgagtttcccgggaaacccgcttttcccgacagccccacaaggggttggtctataaaagaggacgttttccccgtcatcgagattgaagattcttacaggcccatttattcaaattggagttgattcttcttgtctttactttctttctctctttttcttccttttttaatattatcttttgtcaagcctggttccctaagttgaactctcttttcttgtgatcctcctatatagatacgccttgccaaatg

SEQ ID NO: 43 - DNA, 996 bp, Issatchenkia orientalis
  source 1..996: mol_type = genomic DNA
gcggccgctaccataatgtatgcgttgagcctcttgcaccttctttattaggaaatcagttgaaaaatttccggattgtctttattattggcccatttttttttggtcacacctttatttttgtacacttctcgggcaaagcaaaaactatagtaccggataggcctttataaaactccagtgtgtatgattttagttggtgtgccatctacacgttctcttagtttctttatcatgtcacagaaagcaagcatgcaaacccttacaaaaaataacaacatacaaatgcctaaacaactggactataatgatggtgagtcagttacgaaaagagcaagtgggttaatacgatttcgtaagggacagtctgaggaagactacaattttcaaaaggagcagttctggtccacgggtcctttagtacagaatcacacatttgtgactgaatttgttgaaaagtttattgaaaacacaattagtgaagattattcaatcacagatagatcgaaaatagaacgtgaaacaatcatacacggattggagaagctgtattttcaaagggaatatgagcgatgtctaaaagatgttcaactattgaaggacaatatcgataagttcaatcctaatttggatcttaatgaaaagaatttataatgagctgaattatatttcttggatgtgcatcaaaaagatccatgagagtaacgaaaagaaactgggggaaatctaataatttacaatttcaatatacacttctatatcctttaatgtaatggctttataaataaacacgaacttctacagcaccgacgtttctttttcttaccagctcctcttcttcttcttcttcttcttcttcttcttcttcttcttcttcttcttcttcttcttcttcttctttcttaccatcattgccattttccttttttcttatttgctcttgatcctctgttttttcaatttggacaaactcatctaatacaccaacacttttag

(19) United States
(12) Patent Application Publication
(10) Pub. No.: US 2024/0368530 A1
(43) Pub. Date: Nov. 7, 2024

(54) GENETICALLY MODIFIED YEAST AND FERMENTATION PROCESSES FOR THE PRODUCTION OF LACTATE

(71) Applicant: CARGILL, INCORPORATED, Wayzata, MN (US)

(72) Inventors: Keith Michael BRADY, Eden Prairie, MN (US); Hans H. LIAO, Plymouth, MN (US)

(73) Assignee: CARGILL, INCORPORATED, Wayzata, MN (US)

(21) Appl. No.: 18/683,849

(22) PCT Filed: Aug. 3, 2022
(86) PCT No.: PCT/US2022/074468

§ 371 Date: Feb. 15, 2024

Related U.S. Application Data
(60) Provisional application No. 63/234,577, filed on Aug. 18, 2021.

(51) Int. Cl. C12N 1/18; C12N 9/04; C12N 9/12; C12N 9/26; C12P 7/62; C12R 1/84

(52) U.S. Cl. CPC C12N 1/185; C12N 9/0006; C12N 9/1205; C12N 9/2431; C12P 7/62; C12R 2001/84; C12Y 101/01027; C12Y 207/01001; C12Y 207/01004; C12Y 302/01026

(57) ABSTRACT

Disclosed herein are genetically engineered yeast cells capable of producing lactate from sucrose. The genetically engineered yeast cells comprise a polynucleotide encoding an exogenous lactate dehydrogenase enzyme: a polynucleotide encoding an exogenous invertase enzyme: a deletion or disruption of a native pyruvate decarboxylase (PDC) gene: and a genetic modification resulting in overexpression of a native hexokinase gene.

### CROSS-REFERENCE TO RELATED APPLICATIONS

This application claims priority to U.S. Provisional Application No. 63/234,577, filed Aug. 18, 2021, which is incorporated herein by reference in its entirety.

### REFERENCE TO A SEQUENCE LISTING SUBMITTED VIA PATENT CENTER

The content of the Sequence Listing XML file of the sequence listing named “PT-1098-WO-PCR.xml” which is 133 kb in size created on Aug. 1, 2022 and electronically submitted via Patent Center herewith the application is incorporated by reference in its entirety.

### BACKGROUND

Fermentation processes are used commercially at large scale to produce organic molecules such as ethanol, citric acid, and lactic acid. In those processes, a carbohydrate is fed to an organism that is capable of metabolizing it to the desired fermentation product. The carbohydrate and organism are selected together so that the organism is capable of efficiently digesting the carbohydrate to form the product desired in good yield. It is becoming more common to use genetically engineered organisms in these processes, in order to optimize yields and process variables, or to enable particular carbohydrates to be metabolized.

Sucrose is a possible carbohydrate feed for such commercial fermentation processes. However, many organisms are not capable of metabolizing sucrose and/or are not capable of metabolizing the fructose component of sucrose.

### SUMMARY

The present disclosure provides a genetically engineered yeast cell capable of producing lactate from sucrose, the engineered yeast cell comprising a polynucleotide encoding an exogenous lactate dehydrogenase enzyme; a polynucleotide encoding an exogenous invertase enzyme; a deletion or disruption of a native pyruvate decarboxylase (PDC) gene; and a genetic modification resulting in overexpression of a native hexokinase gene. Hexokinase activity in the engineered yeast cell may be higher than hexokinase activity in an equivalent yeast cell lacking the genetic modification. Peak lactate production rate in the engineered yeast cell, when used in a fermentation process in the presence of sucrose, may be higher than peak lactate production rate of an equivalent yeast cell lacking the genetic modification. The genetic modification may comprise replacement of the native hexokinase gene promoter with a constitutive heterologous or artificial promoter. The constitutive heterologous or artificial promoter may be selected from the group consisting of pyruvate decarboxylase (PDC1), glyceraldehyde-3-phosphate dehydrogenase (TDH3), enolase (ENO1), and 3-phosphoglycerate kinase (PGK1). The genetic modification may comprise addition of an exogenous polynucleotide encoding the native hexokinase such that the genetically engineered yeast cell comprises at least one additional copy of a sequence encoding the native hexokinase. The native hexokinase comprises a sequence at least 60%, at least 65%, at least 70%, at least 75%, at least 80%, at least 85%, at least 90%, at least 95%, at least 98%, at least 99%, or 100% identical to at least one of SEQ ID NOs: 24 and 25. The engineered yeast cell may comprise a deletion or disruption of a glycerol-3-phosphate dehydrogenase (GPD) gene. The engineered yeast cell may comprise a deletion or disruption of an L-lactate:cytochrome c oxidoreductase (CYB2) gene. The yeast cell additionally comprises an exogenous polynucleotide encoding a fructokinase. The fructokinase may comprise a sequence at least 60%, at least 65%, at least 70%, at least 75%, at least 80%, at least 85%, at least 90%, at least 95%, at least 98%, at least 99%, or 100% identical to SEQ ID NO: 26. The exogenous polynucleotide encoding the fructokinase may be operably linked to a heterologous or artificial promoter. The heterologous or artificial promoter may be selected from the group consisting of pyruvate decarboxylase (PDC1), glyceraldehyde-3-phosphate dehydrogenase (TDH3), translational elongation factor (TEF), transaldolase (TAL), RPL16B, 3-phosphoglycerate kinase (PGK1), and enolase (ENO1).

The lactate dehydrogenase may comprise a sequence a sequence at least 60%, at least 65%, at least 70%, at least 75%, at least 80%, at least 85%, at least 90%, at least 95%, at least 98%, at least 99%, or 100% identical to at least one of SEQ ID NOs: 30 and 31. The exogenous polynucleotide encoding the lactate dehydrogenase may be operably linked to a heterologous or artificial promoter. The heterologous or artificial promoter may be selected from the group consisting of pyruvate decarboxylase (PDC1), glyceraldehyde-3-phosphate dehydrogenase (TDH3), translational elongation factor (TEF), transaldolase, RPL16B, 3-phosphoglycerate kinase (PGK1), and enolase (ENO1).

The exogenous invertase enzyme may comprise a sequence at least 60%, at least 65%, at least 70%, at least 75%, at least 80%, at least 85%, at least 90%, at least 95%, at least 98%, at least 99%, or 100% identical to at least one of SEQ ID NOs: 12, 33, 34, and 35. The polynucleotide encoding the exogenous invertase enzyme may be operably linked to a heterologous or artificial promoter. The promoter may be selected from the group consisting of pyruvate decarboxylase (PDC1), glyceraldehyde-3-phosphate dehydrogenase (TDH3), translational elongation factor (TEF), transaldolase (TAL), RPL16B, 3-phosphoglycerate kinase (PGK1), and enolase (ENO1).

The yeast cell may be Crabtree negative. The yeast cell may be a yeast of the Issatchenkia orientalis/Pichia fermentans clade. The yeast cell may be an Issatchenkia orientaliscell. The yeast cell may be an Issatchenkia orientalis cell and the native, overexpressed hexokinase comprises a sequence at least 60%, at least 65%, at least 70%, at least 75%, at least 80%, at least 85%, at least 90%, at least 95%, at least 98%, at least 99%, or 100% identical to at least one of SEQ ID NOs: 24 and 25.

The disclosure also provides a genetically engineered Issatchenkia orientalis cell capable of producing lactate from sucrose, the engineered yeast cell comprising a polynucleotide encoding an exogenous lactate dehydrogenase enzyme; a polynucleotide encoding an exogenous invertase enzyme; a deletion or disruption of a native pyruvate decarboxylase (PDC) gene; and a genetic modification resulting in overexpression of a native hexokinase gene, wherein the engineered I. orientalis cell is capable of producing lactate at a titer of at least 30, at least 80, at least 100, or at least 120 g/L.

The disclosure also provides a method for producing lactate from sucrose, the method comprising contacting a substrate comprising sucrose with an engineered yeast cell as described herein, wherein fermentation of the substrate by the engineered yeast produces lactate. The disclosure provides a method for producing lactate from sucrose, the method comprising contacting a substrate comprising sucrose with an engineered yeast cell comprising a polynucleotide encoding an exogenous lactate dehydrogenase enzyme; a polynucleotide encoding an exogenous invertase enzyme; a deletion or disruption of a native pyruvate decarboxylase (PDC) gene; and a genetic modification resulting in overexpression of a native hexokinase gene, wherein fermentation of the substrate by the engineered yeast produces lactate. The volumetric oxygen uptake rate (OUR) may be 0.5 to 40 mmol O₂/(L·h), 1 to 30 mmol O₂/(L·h), 3 to 25 mmol O₂/(L·h), 5 to 20 mmol O₂/(L·h), or 10 to 18 mmol O₂/(L·h). The volumetric OUR may be 10 to 20 mmol O₂/(L·h), 10 to 14 mmol O₂/(L·h), or 15-18 mmol O₂/(L·h). Peak lactate production rate may be at least 5 g L⁻¹ h⁻, at least 6 g L⁻¹ h⁻, at least 7 g L⁻¹ h⁻, or at least 8 g L-1 h-1. Lactate may be produced at a rate of at least 1.5 g L⁻¹ h⁻, at least 2.0 g L⁻¹ h⁻, at least 2.5 g L-1 h-1, at least 3.0 g L⁻¹ h⁻, or at least 3.5 g L⁻¹ h⁻. The fermentation temperature may be in the range of 20° C. to 45° C., 25°° C. to 40° C., or 30°° C. to 38° C. The lactate titer may be at least 30, at least 80, at least 100, or at least 120 g/L.

### BRIEF DESCRIPTION OF THE FIGURES

This patent or application contains at least one drawing executed in color. Copies of this patent or patent application publication with color drawings will be provided by the Office upon request and the payment of the necessary fee.

The drawings illustrate generally, by way of example, but not by way of limitation, various aspects discussed herein.

FIG. 1 shows the production of lactate during the fermentation of strains 1.3 and 1.5a as outlined in Example 2.

FIG. 2 shows the consumption of glucose and fructose and the production of lactate during the fermentation of strains 1.3 and 1.5a as outlined in Example 2.

FIG. 3 shows the average volumetric rate of lactate production during the fermentation of strains 1.3 and 1.5a as outlined in Example 2.

FIG. 4 shows the instantaneous volumetric rate of lactate production during the fermentation of strains 1.3 and 1.5a as outlined in Example 2.

FIG. 5 shows both the average volumetric rate and the instantaneous volumetric rate of lactate production during the fermentation of strains 1.3 and 1.5a as outlined in Example 2.

FIG. 6 shows the production of lactate during the fermentation of strains 1.3 and 1.5a as outlined in Example 3.

FIG. 7 shows glucose and fructose consumption and lactate production during the fermentation of strains 1.3 and 1.5a as outlined in Example 3.

FIG. 8 shows the average volumetric lactate production rate and the instantaneous volumetric lactate production rate during the fermentation of strains 1.3 and 1.5a as outlined in Example 3.

FIG. 9 shows the dextrose consumption and lactate production during fermentation of strains 1.3, 1.6, 1.8a, 1.8b, and 1.8c as outlined in Example 4.

FIG. 10 shows dextrose, fructose, and sucrose consumption during fermentation of strains 1.3, 1.6, 1.8a, 1.8b, and 1.8c as outlined in Example 4.

FIG. 11 shows lactate production during fermentation of strains 1.3, 1.6, 1.8a, 1.8b, and 1.8c as outlined in Example 4.

FIG. 12 shows dextrose consumption and lactate production during fermentation of strains 1.3, 1.7, 1.9a, 1.9b, and 1.9c as outlined in Example 4.

FIG. 13 shows sucrose, dextrose, and fructose consumption during fermentation of strains 1.3, 1.7, 1.9a, 1.9b, and 1.9c as outlined in Example 4.

FIG. 14 shows lactate production during fermentation of strains 1.3, 1.7, 1.9a, 1.9b, and 1.9c as outlined in Example 4.

FIG. 15 shows dextrose consumption and lactate production during fermentation of strains 1.3, 1.10a, 1.10b, and 1.10c as outlined in Example 4.

FIG. 16 shows sucrose, dextrose, and fructose consumption during fermentation of strains 1.3, 1.10a, 1.10b, and 1.10c as outlined in Example 4.

FIG. 17 shows lactate production during fermentation of strains 1.3, 1.10a, 1.10b, and 1.10c as outlined in Example 4.

FIG. 18 shows dextrose consumption and lactate production during fermentation of strains 1.3, 1.11a, 1.11b, 1.11c, 1.11d, 1.11e, and 1.11f as outlined in Example 4.

FIG. 19 shows sucrose, dextrose, and fructose consumption during fermentation of strains 1.3, 1.11a, 1.11b, 1.11c, 1.11d, 1.11e, and 1.11f as outlined in Example 4.

FIG. 20 shows lactate production during fermentation of strains 1.3, 1.11a, 1.11b, 1.11c, 1.11d, 1.11e, and 1.11f as outlined in Example 4.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered to produce lactate which have improved sucrose consumption, in particular, improved fructose consumption. In general, the recombinant cells described herein have a deletion or disruption of a native pyruvate decarboxylase gene and express an exogenous lactate dehydrogenase enzyme and are characterized by at least one of expression of an exogenous invertase enzyme, expression of an exogenous fructose transporter, expression of an exogenous hexokinase gene, and a genetic modification resulting in overexpression of a native hexokinase gene. The disclosure further provides fermentation methods for the production of lactate from sucrose using the genetically engineered cells described herein.

As used herein, “lactate” refers to the salts (2-hydroxypropionate) and acid forms (2-hydroxypropionic acid) of lactic acid. Lactate is measured as the sum of free lactic acid and any lactate salts (excluding the portions attributable to any cation portion of aid salt form present). In other words, “rate of lactate production,” “lactate yield.” “lactate titer,” and the like refer to the rate, yield, titer, respectively of the sum of free lactic acid and any lactate salts.

In general, recombinant cells described herein are yeast cells. Suitable yeast cells may include, but are not limited to, Saccharomyces cerevisiae, Issatchenkia orientalis, Pichia galeiformis, Pichia sp. YB-4149 (NRRL designation), Candida ethanolica, Pichia deserticola, Pichia membranifadens, or Pichia fermentans. An ordinarily skilled artisan would understand the requirements for selection of a suitable yeast cell, and recombinant yeast cells of the present disclosure are not limited to those expressly recited herein.

The recombinant yeast cell may be a Crabtree negative yeast cell. As used herein, “Crabtree negative” refers to a yeast cell that does not exhibit the Crabtree effect of fermentative metabolism under aerobic conditions as a result of the inhibition of oxygen consumption by the microorganism when cultured at high specific growth rates (long-term effect) or in the presence of high concentrations of glucose (short-term effect) due to the inhibition of the synthesis of respiratory enzymes. A Crabtree negative yeast cell will not exhibit this effect and is therefore able to consume oxygen even in the presence of high concentrations of glucose or at high growth rates. Methods for determining whether an organism is Crabtree negative or Crabtree positive are known and described in the art (e.g., See De Deken R.H. (1965) J. Gen. Microbiol., 44:149-156).

The recombinant cell may be a yeast cell of a species within the Issatchenkia orientalis/Pichia fermentans clade. This clade is the most terminal clade that contains at least the species Issatchenkia orientalis, Pichia galeiformis, Pichia sp. YB-4149 (NRRL designation), Candida ethanolica, Pichia deserticola, Pichia membranifadens, and Pichia fermentans. Identification and characterization of species within the I. orientalis/P. fermentans clade is known and described in the art. See, for example, Kurtzman et al. (“Identification and phylogeny of Ascomycetous yeasts from analysis of nuclear large subunit (26S) ribosomal DNA partial sequences,” Antonie van Leeuwanhoek, 73:331-371, 1998) and WO2007032792A2, each of which is incorporated herein by reference. Analysis of the variable D1/D2 domain of the 26S ribosomal DNA from hundreds of ascomycetes has shown that the I. orientalis/P. fermentansclade contains very closely related species. Members of the I. orientalis/P. fermentans clade exhibit greater similarity in the variable D1/D2 domain of the 26S ribosomal DNA to other members of the clade than to yeast species outside of the clade. Therefore, other members of the I. orientalis/P. fermentans clade can be identified by comparison of the D1/D2 domains of their respective ribosomal DNA and comparing to that of other members of the clade and closely related species outside of the clade, using Kurtzman and Robnett's methods (see Kurtzman and Fell, The Yeasts, a Taxonomic Study, Section 35, Issatchenkia Kudryavtsev, pp. 222-223 (1998), which is hereby incorporated by reference). The recombinant cell may be a recombinant Issatchenkia orientalis, Pichia galeiformis, Pichia sp. YB-4149 (NRRL designation), Candida ethanolica, Pichia deserticola, Pichia membranifadens, or Pichia fermentans cell.

When first characterized, the species I. orientalis was assigned the name Pichia kudriavzevii. The anamorph (asexual form) of I. orientalis is known as Candida krusei. Examples of suitable I. orientalis strains may include, but are not limited to, I. orientalis strains ATCC 32196 and I. orientalis strain ATCC PTA-6658.

The recombinant cells described herein include one or more exogenous polynucleotide sequences encoding one or more exogenous polypeptides that, when expressed improve the fermentation of sucrose to lactate by the recombinant cells. The recombinant cell may alternatively or additionally include one or more genetic modifications that increases expression of a native polypeptide, wherein said increase in expression improves the fermentation of sucrose to lactate by the recombinant cell.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactisinvertase gene is exogenous when introduced into I. orientalis.

As used herein, “native” refers to genetic material or an expression product thereof that is found, apart from individual-to-individual mutations which do not affect function or expression, within the genome of wild-type cells of the host cell.

As used herein, the terms “polypeptide” and “peptide” are used interchangeably and refer to the collective primary, secondary, tertiary, and quaternary amino acid sequence and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| | Three | One |
|---|---|---|
| Amino Acid | letter symbol | letter symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the practice of the disclosed pigments, compositions, and methods. Such sequences can be referred to as variants or modified sequences. That is, a polypeptide sequence can be modified yet still retain the ability to exhibit the desired activity. Generally, the variant or modified sequence may include or greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences: Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100: Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA or a genomic DNA sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (i.e., polynucleotides encoding a non-heme iron-binding protein polypeptide) may be codon-optimized for expression in a particular cell including, without limitation, a plant cell, bacterial cell, fungal cell, or animal cell. While polypeptides encoded by polynucleotide sequences found in coral are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as be a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, and/or selection and screening. The native gene to be deleted or disrupted may be replaced with an exogenous nucleic acid of interest for the expression of an exogenous gene product (e.g., polypeptide, enzyme, and the like).

The recombinant cell described herein may have a deletion or disruption of one or more native genes encoding an enzyme involved in ethanol fermentation or consumption. Deletion or disruption of one or more of these ethanol biosynthetic pathway enzymes decreases the ability of the cell to product ethanol, thereby increasing fermentation production of lactate.

The recombinant cells described herein include a deletion or disruption of a native pyruvate decarboxylase (PDC) gene. The native PDC gene encodes an enzyme that catalyzes the conversation of pyruvate to acetaldehyde and carbon dioxide. When the host cell contains multiple PDC genes, it is preferred to delete or disrupt at least one of them and more preferred to disrupt all of them to more completely eliminate the host cell's ability to produce ethanol. When the recombinant cell is an I. orientalis cell, the recombinant cell may comprise a deletion or disruption of a PDC gene encoding an amino acid sequence at least 60%, at least 65%, at least 70%, at least 75%, at least 80%, at least 85%, at least 90%, at least 95%, at least 98%, at least 99%, or 100% identical to SEQ ID NO:32. Methods for the deletion or disruption of the PDC genes of I. orientalis are known and described in the art. See, for example, WO2007032792A2 and WO2017091610A1, which are incorporated herein by reference.

The recombinant cells described herein may include a deletion or disruption of a native glycerol-3-phosphate dehydrogenase (GPD) gene. Deletion or disruption of a native GPD gene improves acetate consumption by providing the cell with a greater pool of reducing equivalents to assist in the oxido-reduction of acetate to ethanol. When the ethanol biosynthetic pathway is disrupted, this increased pool of reducing equivalents can improve production of a fermentation product, e.g., lactate. When the host cell contains multiple GPD genes, it is preferred to delete or disrupt at least one of them and more preferred to disrupt all of them.

The recombinant cells described herein may include a deletion or disruption of a native L-lactate:cytochrome c oxidoreductase gene (CYB2). When the host cell contains multiple CYB2 genes, it is preferred to delete or disrupt at least one of them and more preferred to disrupt all of them. For example, I. orientalis includes two alleles of the CYB2 gene (CYB2a and CYB2b) and both may be deleted or disrupted when I. orientalis is used as the host cell.

The recombinant cells described herein may include one or more genetic modifications in which an exogenous nucleic acid is integrated into the genome of the host cell. One of skill in the art know how to select suitable loci in a vest genome for integration of the exogenous nucleic acid. For example, in an I. orientalis host cells, suitable interaction loci may include, but are not limited to, the ('YB2B loci (defined as the loci flanked by SEQ ID NO:38 and SEQ ID NO:39), the ato2 loci (defined as the loci flanked by SEQ ID NO:40 and SEQ ID NO:41), and the adh9091 loci (defined as the loci flanked by SEQ ID NO:42 and SEQ ID NO:43). Other suitable integration loci may be determined one of skill in the art. Furthermore, one of skill in the art would recognize how to use sequences to design primers to verify correct gene integration at the chosen locus.

The recombinant cells described herein are capable of producing lactate and include at least one functional, exogenous lactate dehydrogenase (LDH) enzyme. The LDH enzyme may be expressed from an exogenous nucleic acid on a plasmid in the recombinant cell or an exogenous nucleic acid encoding the LDH may be integrated into the genome of the recombinant host cell. The LDH enzyme may be any suitable enzyme with lactate dehydrogenase activity. As used herein, “lactate dehydrogenase activity” refers to the ability catalyze the reaction of pyruvate to lactate. Suitable LDH enzymes may include, but are not limited to, enzymes categorized under Enzyme Commission (EC) numbers 1.1.1.27 (L-lactate dehydrogenases) and 1.1.1.28 (D-lactate dehydrogenases). The LDH enzyme may be from any suitable organism or may be synthetic. Suitable LDH enzymes may be the LDH enzymes from Lactobacillus helveticus, Lactobacillus casei, Bacillus megaterium, Pediococcus acidilactici, Rhizopus oryzae, Bos taurus, and the like.

The LDH enzyme may be a Lactobacillus helveticus L-LDH (SEQ ID NO:30). A related L. helveticus LDH is provided at UniProt Accession No. 032765 (SEQ ID NO:31). The recombinant cell capable of producing lactate may include an LDH enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 30 and 31. The recombinant cell capable of producing lactate may include an exogenous nucleic acid encoding an LDH enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 30 and 31. Recombinant cells capable of producing lactate and comprising a gene encoding the LDH of SEQ ID NO:31 were previously described in WO2007032792, which is incorporated herein by reference in its entirety.

The recombinant cells described herein may include an exogenous nucleic acid encoding an invertase enzyme. The invertase enzyme may be expressed from an exogenous nucleic acid on a plasmid or an exogenous nucleic acid integrated into the genome of the recombinant host cell. The invertase enzyme may be any suitable enzyme with invertase activity. As used herein, “invertase activity” refers to the ability to catalyze the hydrolysis of sucrose to fructose and glucose. Suitable invertase enzymes may include, but are not limited to, enzymes categorized under EC number 3.2.1.26. The invertase may be from any suitable organism or may be synthetic. Suitable invertase enzymes may be invertase enzymes from Kluyveromyces lactis (SEQ ID NO:12), Saccharomyces cerevisiae (SEQ ID NO:33), Schizosaccharomyces pombe (SEQ ID NO:34), Aspergillus niger (SEQ ID NO:35), and the like. The recombinant cell may include an invertase enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 12, 33, 34, and 35. The recombinant cell capable of producing lactate may include an exogenous nucleic acid encoding an invertase enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 12, 33, 34, and 35.

The recombinant cells described herein may include an exogenous nucleic acid encoding a fructokinase. The fructokinase enzyme may be expressed from an exogenous nucleic acid on a plasmid or an exogenous nucleic acid integrated into the genome of the recombinant host cell. The fructokinase enzyme may be any suitable enzyme with fructokinase activity. As used herein, “fructokinase activity” refers to the ability to catalyze the transfer of a phosphate group from adenosine triphosphate (ATP) to fructose. Suitable fructokinase enzymes may include, but are not limited to, enzymes categorized under EC number 2.7.1.4. The fructokinase may be from any suitable organisms or may be synthetic. For example, the fructokinase enzyme may the fructokinase enzyme from Clostridium acetobutylicum (UniProt Ref. Q9L8G5; SEQ ID NO:26). The recombinant cell may include a fructokinase enzyme with a sequence at least 60%, at least 65%, at least 70%, at least 75%, at least 80%, at least 85%, at least 90%, at least 95%, at least 98%, at least 99%, or 100% identical to SEQ ID NO: 26. The recombinant cell may include an exogenous nucleic acid encoding a fructokinase enzyme with a sequence at least 60%, at least 65%, at least 70%, at least 75%, at least 80%, at least 85%, at least 90%, at least 95%, at least 98%, at least 99%, or 100% identical to SEQ ID NO:26.

The recombinant cells described herein may include an exogenous nucleic acid encoding a native or exogenous hexokinase or may have a genetic modification resulting in overexpression of a native hexokinase. The hexokinase may be any suitable enzyme with hexokinase activity. As used herein, “hexokinase activity” refers to the ability to catalyze the phosphory lation of a hexose by ATP to a hexose phosphate. For example, the hexokinase enzyme may catalyze the addition of a phosphate from ATP to glucose to form glucose-6-phosphate. Suitable hexokinase enzymes may include, but are not limited to, enzymes categorized under EC number 2.7.1.1. The hexokinase may be a hexokinase I, hexokinase II, or hexokinase III isozyme. The hexokinase may a hexokinase native to host cell or the hexokinase may be an exogenous hexokinase. For example, when the host organism is I. orientalis, the hexokinase may be an enzyme with a sequence at least 60%, at least 65%, at least 70%, at least 75%, at least 80%, at least 85%, at least 90%, at least 95%, at least 98%, at least 99%, or 100% identical to at least one of SEQ ID NOs: 24 and 25. The recombinant cell may comprise an exogenous nucleic acid encoding a hexokinase at least 60%, at least 65%, at least 70%, at least 75%, at least 80%, at least 85%, at least 90%, at least 95%, at least 98%, at least 99%, or 100% identical to at least one of SEQ ID NOs: 24 and 25. The recombinant cell may include a genetic modification that increases expression of a hexokinase at least 60%, at least 65%, at least 70%, at least 75%, at least 80%, at least 85%, at least 90%, at least 95%, at least 98%, at least 99%, or 100% identical to at least one of SEQ ID NOs: 24 and 25. The genetic modification may include, but is not limited to, insertion of additional copies of a nucleic acid encoding the native hexokinase into the cell, insertion of a constitutive promoter upstream of the coding region of the native hexokinase gene in the genome of the host cell, and/or modification of the existing promoter upstream of the coding region of the native hexokinase gene in the genome of the host cell. One of skill in the art will recognize that expression of a native hexokinase gene may be increased by a number of methods known in the art and will be able to select and apply such methods as appropriate.

The recombinant cells described herein may include an exogenous nucleic acid encoding a fructose transporter. The fructose transporter may be expressed from an exogenous nucleic acid on a plasmid or an exogenous nucleic acid integrated into the genome of the recombinant host cell. The fructose transporter may be any suitable enzyme with fructose transporter activity. As used herein, “fructose transporter activity” refers to the ability to catalyze the transfer of fructose through a plasmid membrane. Enzymes with fructose transporter activity may also be referred to in the art as transferase, symporters, facilitators, and the like. The fructose transporter may be from any suitable organism or may be synthetic. For example, the fructose transporter may be a fructose transporter from K. lactis (UniProt Ref. F2Z6G6, SEQ ID NO:27), Zygosaccharomyces bailii (UniProt Ref. Q70WR7, SEQ ID NO:28), Saccharomyces carlsbergensis (GenBank Ref. EHN03988.1; SEQ ID NO:29), Botryotinia fucleliana (UniProt Ref. Q5XTQ5; SEQ ID NO:36), or Ganoderma boninense (GenBank Ref. VWO95402.1; SEQ ID NO: 37). The recombinant cell may include a fructose transporter with a sequence at least 60%, at least 65%, at least 70%, at least 75%, at least 80%, at least 85%, at least 90%, at least 95%, at least 98%, at least 99%, or 100% identical to one of SEQ ID NOs: 27, 28, 29, 36, and 37. The recombinant cell may include an exogenous polynucleotide encoding a fructose transporter with a sequence at least 60%, at least 65%, at least 70%, at least 75%, at least 80%, at least 85%, at least 90%, at least 95%, at least 98%, at least 99%, or 100% identical to ate least one of SEQ ID NOs: 27, 28, 29, 36, and 37. A recombinant cell including a fructose transporter as described herein will have an increased rate of fructose consumption (e.g., fructose uptake by the cell and/or fermentation of fructose to one or more end products) compared to an equivalent cell lacking said fructose transporter. Similarly, a recombinant cell including a fructose transporter as described herein will have a glucose consumption rate that is equivalent or greater than the glucose consumption rate in an equivalent cell lacking said fructose transporter. In other words, while the presence of the fructose transporter increases the rate of fructose consumption, it will not decrease the rate of glucose consumption.

The exogenous nucleic acids in the recombinant cells described herein may be under the control of a promoter. For example, the exogenous nucleic acid may be operably linked to a heterologous or artificial promoter. Suitable promoters are known and described in the art. Promoters may include, but are not limited to, RPL16B (SEQ ID NO: 14), pyruvate decarboxylase (PDC1; SEQ ID NO:13), glyceraldehyde-3-phosphate dehydrogenase (TDH3; SEQ ID NO:17), translational elongation factor (TEF; SEQ ID NO:18), transaldolase (TAL; SEQ ID NO:16), enolase (ENO1; SEQ ID NO: 19), 3-phosphoglycerate kinase (PGK1; SEQ ID NO:15).

The exogenous nucleic acids in the recombinant cells described herein may be under the control of a terminator. For example, the exogenous nucleic acid may be operably linked to a heterologous or artificial terminator. Suitable terminators are known and described in the art. Terminators may include, but are not limited to, PDC (SEQ ID NO:20), and ScGAL10 (SEQ ID NO: 21).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding the thermolabile non-heme iron-binding polypeptide. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods for the production of lactate using the recombinant cells described herein. The fermentation methods include the step of fermenting a substrate using the genetically engineered yeasts described herein to product lactate. The fermentation method can include additional steps, as would be understood by a person skilled in the art. Non-limiting examples of additional process steps include maintaining the temperature of the fermentation broth within a predetermined range, adjusting the pH during fermentation, and isolating the lactate from the fermentation broth. The fermentation process may be a microaerobic process.

The fermentation method can be run using sucrose as a substrate, as a result of the genetic modifications to the recombinant cell described herein. The substrate of the fermentation method can also include other components in addition to sucrose. The fermentation substrate can also include glucose, xylose, fructose, hydrozylates of starch, lignocellulosic hydrozylates, or a combination thereof. As contemplated herein, the sucrose component of the substrate will be hydrolyzed into glucose and fructose via the activity of an invertase and/or sucrase. Accordingly. the fermentation substrate may not contain any sucrose per se because all of the sucrose may be hydrolyzed at some point during the process.

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25° C. to 45° C., 20° C. to 40° C., or 33°° C. to 38° C. However, a skilled artisan will recognize that the fermentation temperature is not limited to any specific range recited herein and may be modified as appropriate.

The fermentation process can be run within certain oxygen uptake rate (OUR) ranges. The volumetric OUR of the fermentation process can be in the range of 0.5 to 40, 1 to 30, 3 to 25, 5 to 20, or 10 to 18 mmol O₂/(L·h). In some embodiments, the specific OUR can be in the range of 0.2 to 13, 0.3 to 10, 1 to 7, or 2 to 6 mmol O₂/(g cell dry weight·h). However, the volumetric or specific OURs of the fermentation process are not limited to any specific rates or ranges recited herein.

The fermentation process can be run at various cell concentrations. In some embodiments, the cell dry weight at the end of fermentation can be 1 to 20, 1 to 10, 2 to 8, or 2.5 to 6 g cell dry weight/L. Further, the pitch density or pitching rate of the fermentation process can vary. In some embodiments, the pitch density can be 0.05 to 5, 0.05 to 4, or 0.05 to 2 g cell dry weight/L.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

The lactate production rate of the process may be at least 1.0, at least 1.5, or at least 2.0, at least 2.5, at least 3.0, or at least 3.5 g L⁻¹ h⁻. The lactate yield of the process may be at least 55 percent, at least 65 percent, at least 70 percent, at least 75 percent, at least 80 percent, or at least 85 percent. The final lactate titer of the process may be at least 30, at least 80, at least 100, or at least 120 g/liter. The fermentation process may have a peak lactate production rate of at least 5, at least 6, at least 7, or at least 8 g L⁻¹ h⁻.

In some aspects, the fermentation process can include sucrose as a substrate for only a portion of the process. For example, the fermentation process can include the step of generating a yeast seed culture using sucrose as substrate, then running the full production batch with a hydrolysate, a hydrolysate supplemented with sucrose, or other substrate instead of sucrose. The fermentation process can be run as a sucrose-fed batch. Further, the fermentation process can be a batch process, continuous process, or semi-continuous process, as would be understood by a person skilled in the art.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1: Genetically Modified Yeast Strains

### Strain 1-1

Strain E described by Brady et al. (International Application Publication No. WO2015195934A2, published Dec. 23, 2015) is an Issachenkia orientalis host strain selected for improved lactate productivity in the presence of increased concentrations of free lactate, evolved from ATCC PTA-6658, in which both alleles of the pyruvate decarboxylase (PDC) gene are knocked out and replaced with lactate dehydrogenase (LDH) from Lactobacillus helveticus, and both alleles of both of the L-lactate:cytochrome c oxidoreductase genes (CYB2a and CYB2b) and the glyceraldehyde-3-phosphate dehydrogenase gene (GPD) are deleted. As referred to herein, Strain 1-1 refers to Strain E as described by Brady et al. in International Application Publication No. WO2015195934A2, published Dec. 23, 2015, which is incorporated herein by reference in its entirety.

### Strain 1-1a

Strain 1-1 was subjected to several rounds of mutagenesis and selection in the presence of increased concentrations of free lactate to identify strains with improved lactate productivity. Resulting isolates were streaked for single colony isolation on YPD plates. A single colony was selected and designated strain 1-1a.

### Strain 1-2

Strain 1-1a was transformed with SEQ ID NO:2. SEQ ID NO:2 is segment of SEQ ID NO: 1, a plasmid containing the invertase gene from K. lactis (KlINV; SEQ ID NO:11) encoding the amino acid sequence of SEQ ID NO: 12. SEQ ID NO:2 contains i) an expression cassette for KlINV, encoding the amino acid sequence SEQ ID NO:12, under the control of the RPL16b promoter SEQ ID NO:14 and the PDC terminator SEQ ID NO:20; and ii) flanking DNA for targeted chromosomal integration into the CYB2B loci. Transformants are selected on YNB+Sucrose plates. Resulting transformants are streaked for single colony isolation on YNB+Sucrose plates. A single colony is selected. Correct integration of SEQ ID NO:2 into the selected colony is verified by PCR. A PCR verified isolate is designated Strain 1-2.

### Strain 1-3

Strain 1-2 was transformed with SEQ ID NO:3. SEQ ID NO:3 contains i) an expression cassette for the selectable marker gene melibiase from S. cerevisiae (ScMEL5) including the PGK promoter (SEQ ID NO: 15) and the MEL5 terminator (SEQ ID NO:22) and flanked by LoxP sites; ii) an expression cassette for KlINV, encoding the amino acid sequence SEQ ID NO:12, under the control of the RPL16b promoter SEQ ID NO: 14 and the PDC terminator SEQ ID NO:20; and iii) flanking DNA for targeted chromosomal integration into the CYB2B loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. A single blue colony was selected. Correct integration of SEQ ID NO:3 into the selected blue colony was verified by PCR. A PCR verified isolate was designated Strain 1-3.

### Strain 1-4

Removal of the ScMELS selectable marker from Strain 1-3 was accomplished using a Cre-LoxP recombinase system. This system includes two 34 bp regions, called loxP sites, made up of two 13bp inverted repeats separated by an 8bp spacer region derived from Bacteriophage P1. LoxP sites in Strain 1-3 are located directly upstream and downstream of the ScMELS selectable marker. Recombination at the loxP sites is facilitated by the overexpression of a recombinase gene (Cre).

Strain 1-3 was transformed with SEQ ID NO:23. SEQ ID NO:23 contains: i) an expression cassette for the selectable marker gene invertase from S. cerevisiae (ScSUC2); and ii) an expression cassette for CRE recombinase gene (Cre) to recycle the selectable marker ScMEL5. Transformants were selected on YNB plates containing 2% sucrose as sole carbon source and 32 μg/mL x-alpha-gal which provides colorimetric indication of the absence of the ScMEL5 marker gene. Resulting transformants were streaked for single colony isolation on YPD containing 32 μg/mL x-alpha-gal. A single white colony was selected. Loss of ScMELS from the selected white colony was verified by PCR. A PCR verified isolate is designated Strain 1-4.

### Strains 1-5a and 1-5b

Strain 1-4 was transformed with SE ID NO: 4. SEQ ID NO:4 contains i) an expression cassette for the selectable marker gene melibiase from S. cerevisiae (ScMEL5) including the PGK promoter SEQ ID NO: 15 and the MEL5 terminator SEQ ID NO:22 and flanked by LoxP sites; ii) an expression cassette for the hexokinase gene from I. orientalis, encoding the amino acid sequence SEQ ID NO:25, under the control of the PDC promoter SEQ ID NO: 13 and the S. cerevisiae GAL10 terminator SEQ ID NO:21; and iii) flanking DNA for targeted chromosomal integration into the ato2 loci. Transformants were selected on YNB plates containing 2% melibiose as sole carbon source and 32 μg/mL x-alpha-gal (5-bromo-4-chloro-3-indoxyl-α-D-galactopyranoside) which provides colorimetric indication of the presence of the ScMEL5 marker gene. Resulting transformants were streaked for single colony isolation on YPD containing 32 μg/mL x-alpha-gal. Two single blue colonies were selected. Correct integration of SEQ ID NO: 4 into the selected blue colonies was verified by PCR. PCR verified isolates were designated Strains 1-5a and 1-5b.

### Strain 1-6

Strain 1-4 was transformed with SEQ ID NO:5. SEQ ID NO:5 contains i) an expression cassette for the selectable marker gene melibiase from S. cerevisiae (ScMEL5) including the PGK promoter SEQ ID NO:15 and the MEL5 terminator SEQ ID NO:22 and flanked by LoxP sites; ii) an expression cassette for the fructokinase gene from Clostridium acetobutylicum (CaScrK), encoding the amino acid sequence SEQ ID NO:26, under the control of the PDC promoter SEQ ID NO: 13 and the S. cerevisiae GAL10 terminator SEQ ID NO:21; and iii) flanking DNA for targeted chromosomal integration into the ato2 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. A single blue colony was selected. Correct integration of SEQ ID NO:5 into the selected blue colony was verified by PCR. A PCR verified isolate was designated Strain 1-6.

### Strain 1-7

Strain 1-4 was transformed with SEQ ID NO:6. SEQ ID NO:6 contains i) an expression cassette for the selectable marker gene melibiase from S. cerevisiae (ScMEL5) including the PGK promoter SEQ ID NO:15 and the MEL5 terminator SEQ ID NO:22 and flanked by LoxP sites; ii) an expression cassette for the fructose transporter gene from Kluyveromyces lactis (KlFrt1), encoding the amino acid sequence SEQ ID NO:27, under the control of the PDC promoter SEQ ID NO: 13 and the S. cerevisiae GAL10 terminator SEQ ID NO:21; and iii) flanking DNA for targeted chromosomal integration into the ato2 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. A single blue colony was selected. Correct integration of SEQ ID NO:6 into the selected blue colony was verified by PCR. A PCR verified isolate was designated Strain 1-7.

### Strains 1-8a, 1-8b, and 1-8c

Strain 1-4 was transformed with SEQ ID NO:7. SEQ ID NO:7 contains i) an expression cassette for the selectable marker gene melibiase from S. cerevisiae (ScMEL5) including the PGK promoter SEQ ID NO: 15 and the MEL5 terminator SEQ ID NO:22 and flanked by LoxP sites; ii) an expression cassette for the fructokinase gene from Clostridium acetobutylicum (CaScrK), encoding the amino acid sequence SEQ ID NO:26, under the control of the PDC promoter SEQ ID NO: 13 and the S. cerevisiae GAL10 terminator SEQ ID NO:21; and iii) flanking DNA for targeted chromosomal integration into the adh9091 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. Three single blue colonies were selected. Correct integration of SEQ ID NO:7 into the selected blue colonies was verified by PCR. PCR verified isolates were designated Strains 1-8a, 1-8b, and 1-8c.

### Strains 1-9a, 1-9b, and 1-9c

Strain 1-4 was transformed with SEQ ID NO:8. SEQ ID NO:8 contains i) an expression cassette for the selectable marker gene melibiase from S. cerevisiae (ScMEL5) including the PGK promoter SEQ ID NO: 15 and the MEL5 terminator SEQ ID NO:22 and flanked by LoxP sites; ii) an expression cassette for the fructose transporter gene from Kluyveromyces lactis (KlFrt1), encoding the amino acid sequence SEQ ID NO:27, under the control of the PDC promoter SEQ ID NO: 13 and the S. cerevisiae GAL10 terminator SEQ ID NO:21; and iii) flanking DNA for targeted chromosomal integration into the adh9091 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. Three single blue colonies were selected. Correct integration of SEQ ID NO:8 into the selected blue colonies was verified by PCR. PCR verified isolates were designated Strains 1-9a, 1-9b, and 1-9c.

### Strains 1-10a, 1-10b, and 1-10c

Strain 1-4 was transformed with SEQ ID NO:9. SEQ ID NO:9 contains i) an expression cassette for the selectable marker gene melibiase from S. cerevisiae (ScMEL5) including the PGK promoter SEQ ID NO: 15 and the MEL5 terminator SEQ ID NO:22 and flanked by LoxP sites; ii) an expression cassette for the fructose transporter gene from Zygosaccharomyces bailii (ZbFfz1), encoding the amino acid sequence SEQ ID NO:28, under the control of the PDC promoter SEQ ID NO: 13 and the S. cerevisiae GAL10 terminator SEQ ID NO:21; and iii) flanking DNA for targeted chromosomal integration into the adh9091 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. Three single blue colonies were selected. Correct integration of SEQ ID NO:9 into the selected blue colonies was verified by PCR. PCR verified isolates were designated Strains 1-10a, 1-10b, and 1-10c.

### Strains 1-11a, 1-11b, 1-11c, 1-11d, 1-11e, and 1-11f

Strain 1-4 was transformed with SEQ ID NO:10. SEQ ID NO:10 contains i) an expression cassette for the selectable marker gene melibiase from S. cerevisiae (ScMEL5) including the PGK promoter SEQ ID NO: 15 and the MEL5 terminator SEQ ID NO:22 and flanked by LoxP sites; ii) an expression cassette for the fructose transporter gene from Saccharomyces carlsbergensis (ScarlFsy1), encoding the amino acid sequence SEQ ID NO:29, under the control of the PDC promoter SEQ ID NO: 13 and the S. cerevisiae GAL10 terminator SEQ ID NO:21; and iii) flanking DNA for targeted chromosomal integration into the adh9091 loci. Transformants were selected on YNB plates containing 2% melibiose as sole carbon source and 32 μg/mL x-alpha-gal which provides colorimetric indication of the presence of the ScMEL5 marker gene. Resulting transformants were streaked for single colony isolation on YPD containing 32 μg/mL x-alpha-gal. Six single blue colonies were selected. Correct integration of SEQ ID NO:9 into the selected blue colonies was verified by PCR. PCR verified isolates were designated Strains 1-11a, 1-11b, 1-11c, 1-11d, 1-11e, and 1-11f.

### Example 2: Fermentations with lactate producing strains 1.3 and 1.5a

The lactate producing yeast strains 1.3 and 1.5a were run in fermenters to assess glucose and fructose consumption as well as lactate production. Strains 1.3 and 1.5a were streaked out for single colonies on a YPD plate (bacteriological peptone 20 g/L, yeast extract 10 g/L, glucose 20 g/L, and agar 15 g/L) and incubated until single colonies were visible. Incubation at room temperature resulted in single colony growth in about 72 hours and incubation at 30° C. resulted in single colony growth in about 18 hours. Cells from plates were scraped into sterile seed medium (Table 2) and the optical density (OD₆₀₀) was measured. Optical density is measured at a wavelength of 600 nm with a 1 cm path length cuvette using a model Genesys20 spectrophotometer (Thermo Scientific). Seed cultures are run in 500 mL shake flasks containing 50 mL seed medium at 34° C. and 250) RPM until they reach an OD₆₀₀ of 4-8.

A 2L capacity fermenter is inoculated with the seed culture to reach on initial OD₆₀₀ of 0.2. Separate fermenters are inoculated with seed cultures for each of the strains. Immediately prior to inoculating, 1.45 L of fermentation medium is added to the fermenter. The fermentation medium is sterilized, pH to 5.5, and contains the components outlined in Table 4.

pH in the fermenters starts at 5.5 and free falls to 4.45 where it is maintained by controlled addition of a 30% suspension of lime (calcium hydroxide) until 84 g of the lime suspension has been added, after which no further pH control occurs. The fermenters are sparged with 0.25 SLPM (standard liters per minute) air through a sparge ring at the base of the vessel. An oxygen uptake rate of 12-13 mmol O₂/(L*h) is achieved by selecting an appropriate agitation speed, approximately 600 RPM±25. These fermentations are operated such that after the cells achieve a sufficient density, oxygen limitation is achieved and subsequently maintained throughout the rest of the fermentation (e.g., dissolved oxygen less than about 10.) Dissolved oxygen is measured using Mettler Toledo InProR® 6800 sensor (Mettler-Toledo GmbH, Urdorf, Switzerland), calibrated prior to inoculation. 0% is calibrated by unplugging the probe and measuring a null signal, 100% is calibrated using air sparging according to the run conditions in the vessel as detailed above (prior to inoculation). Oxygen uptake rate (“OUR”) is calculated using methods known to those in the art as described above. For this example, Oxygen, N₂ and CO₂ values are measured by a mass spectrometer.

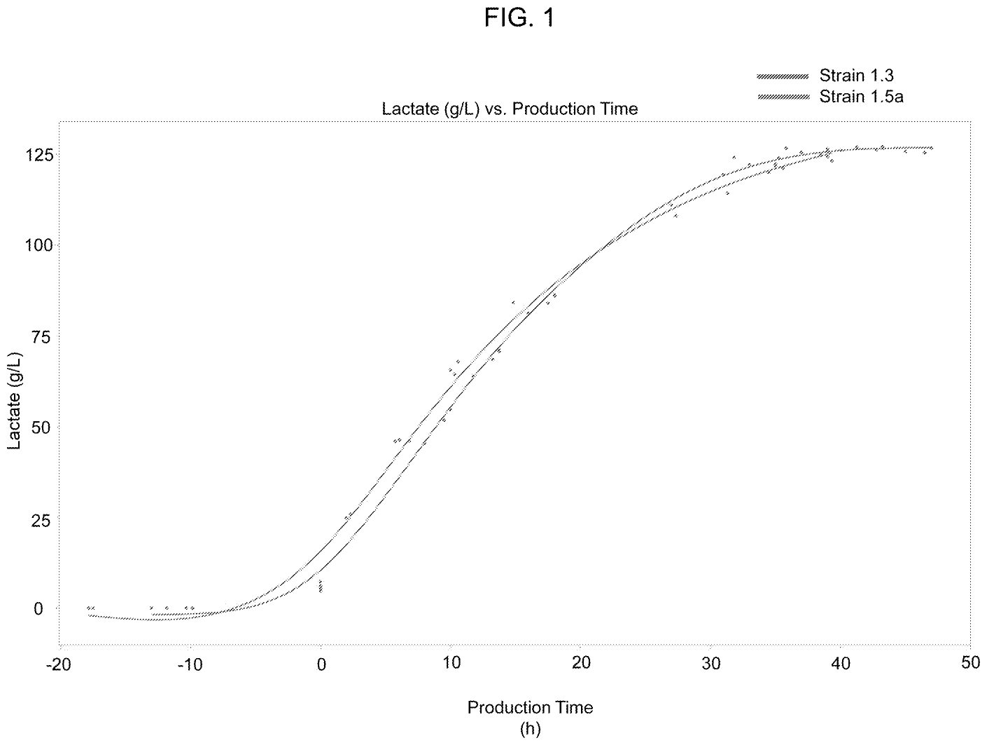

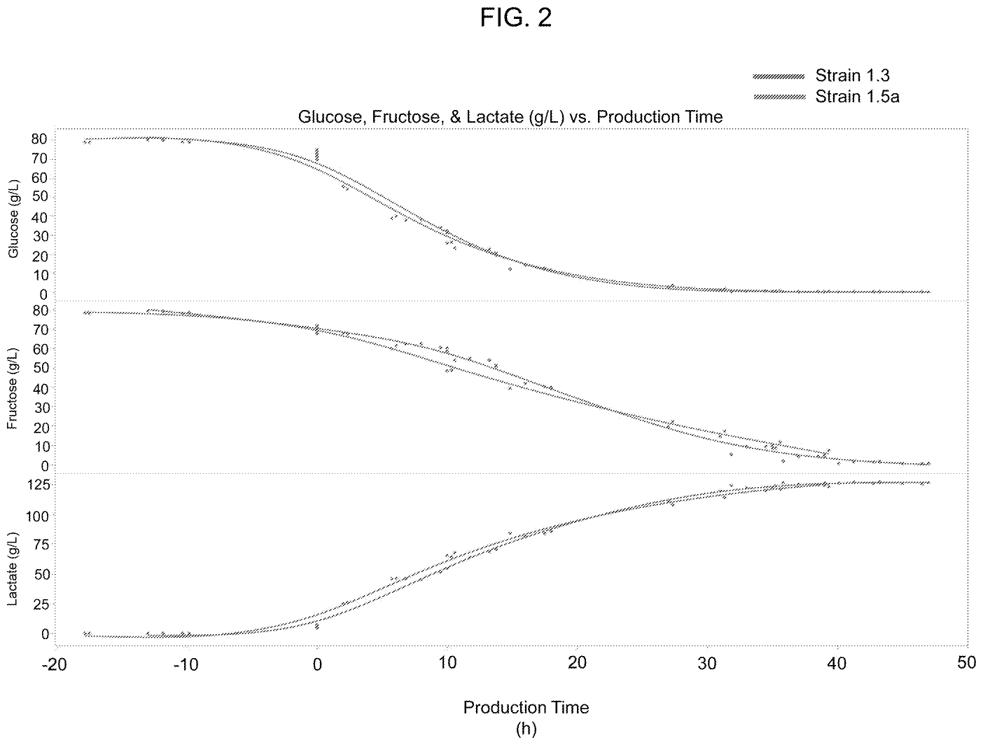

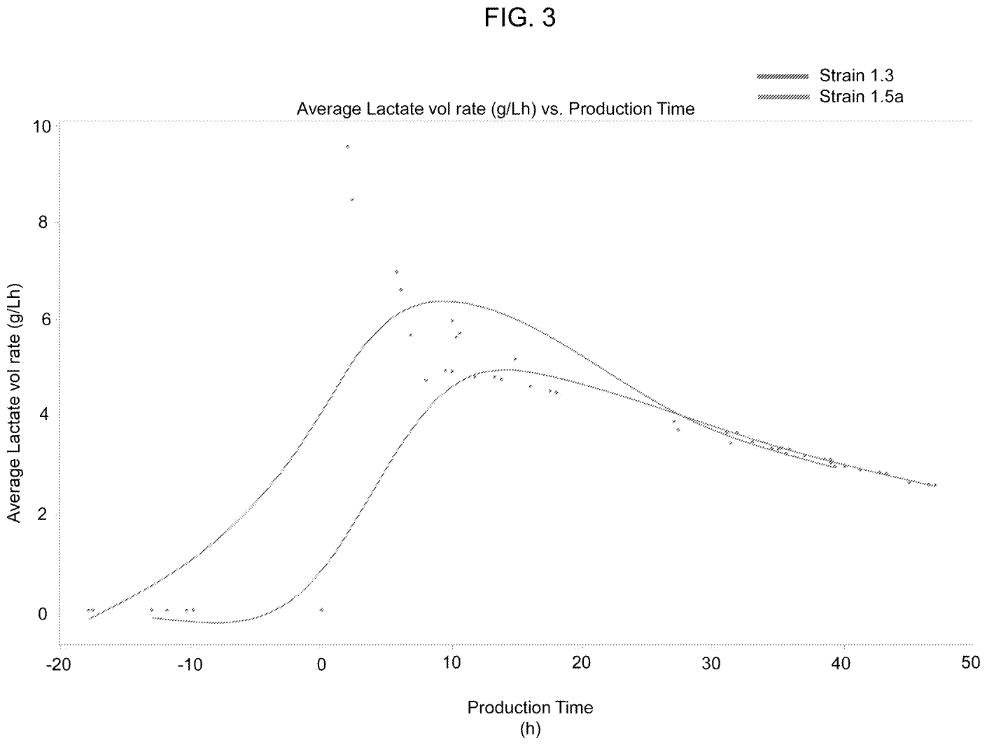

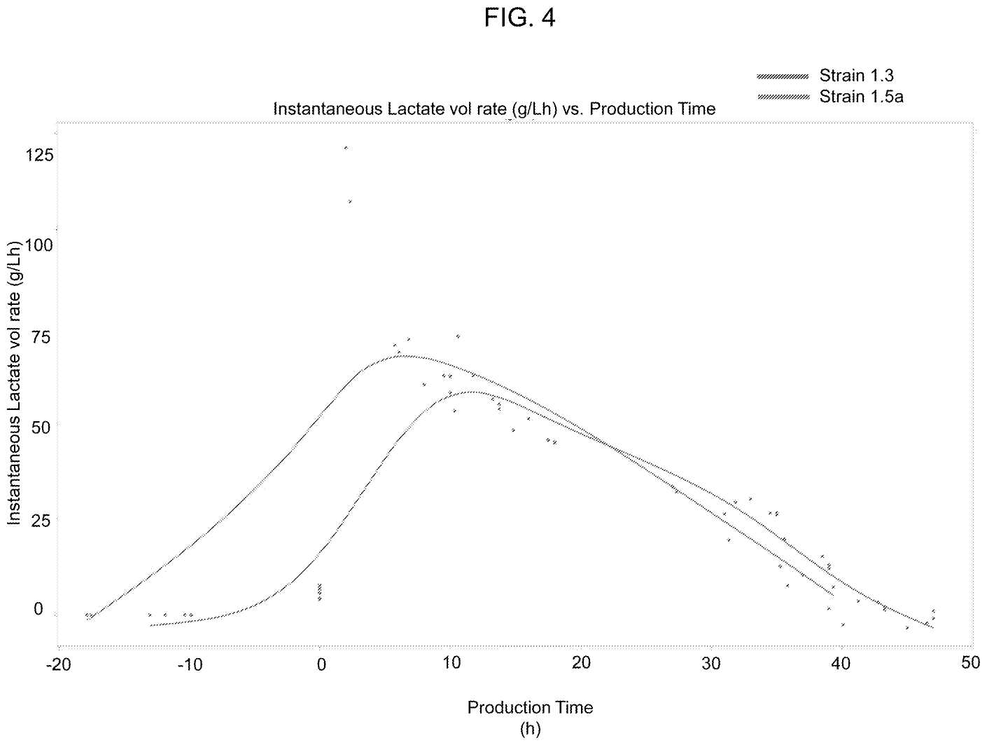

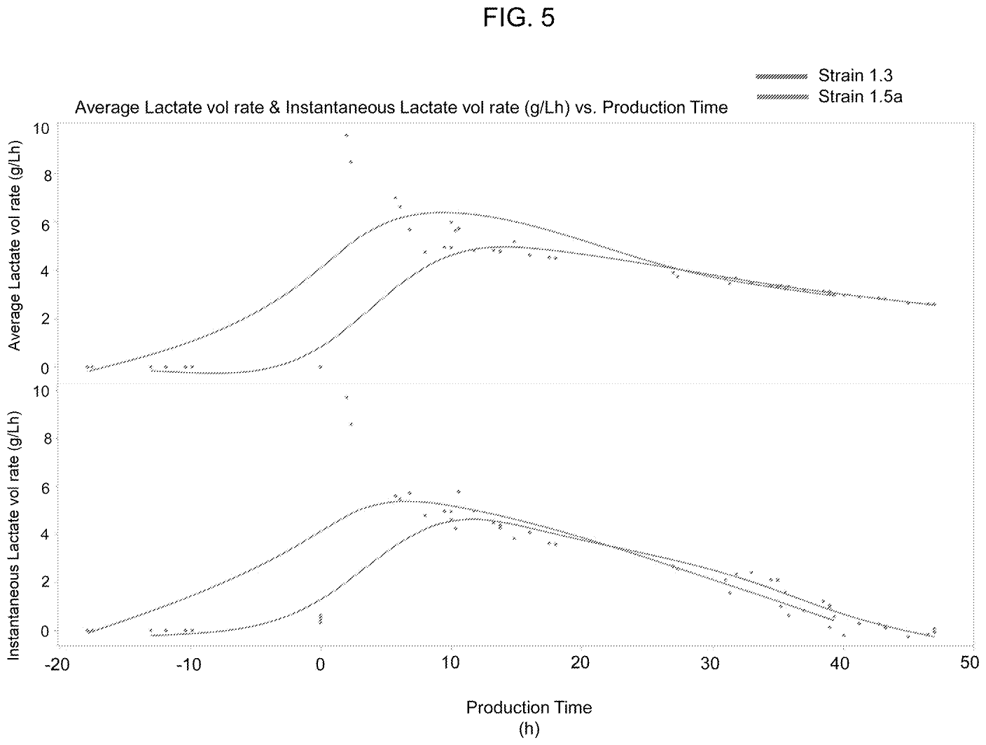

Samples are taken immediately after inoculation, at the end of the batch, and periodically throughout the fermentation. Samples are analyzed for lactate, glucose, fructose, sucrose, and arabitol concentration by high performance liquid chromatography with refractive index detector. Fermentation results are reported in Table 6 and in FIGS. 1-5.

Overall, strain 1.5a, in which I. orientalis hexokinase 2-1 (HXK2-1) is over expressed, had a higher rate of lactate production and a higher peak rate of lactic production than the control stain 1.3.

TABLE 2
Seed Medium

| | Amount in 1000 | |
|---|---|---|
| Component | mL final volume | |
| Dextrose anhydrous | 100 | g |
| MES hydrate | 19.5 | g |
| DMu salts 25× (Table 3) | 40 | mL |
| D-Biotin Stock Solution | 1 | mL |
| (1000×, 1.04 g/mL) | | |
| Trace Composition (Table 5) | 1 | mL |
| Lubrizol 1:100 | 1 | mL |
| Glycerol 10% | 1 | mL |

TABLE 3
DMu salts 25×

| Chemical | Molecular Weight | g/L |
|---|---|---|
| Urea | 60.06 | 57.0 |
| Monopotassium Phosphate | 136.09 | 75.0 |
| Magnesium Sulfate | 246.47 | 12.5 |
| Heptahydrate | | |

TABLE 4
Fermentation Medium

| Component | Concentration | |
|---|---|---|
| Sucrose | 156 | g/L |
| Lubrizol 1:100 | 1.07 | mL/L |
| Glycerol 10% | 1.07 | mL/L |
| APK | 1.15 | mL/L |
| (11.7% (w/v) anhydrous KOH, | | |
| 30.3% (w/v) anhydrous phosphoric acid, | | |
| 4.86% (w/v) anhydrous ammonia) | | |
| 50% Urea | 1.72 | mL/L |
| Magnesium Sulfate | 1.00 | mL/L |
| (1000× stock solution at 125 g/L) | | |
| Trace Composition (Table 2.4) | 1.00 | mL/L |
| D-Biotin Stock Solution | 1.00 | mL/L |
| (1000×, 1.04 g/mL) | | |

TABLE 5
Trace Composition

| Chemical | Formula | Molecular Weight | g/L |
|---|---|---|---|
| EDTA (disodium salt | C₁₀H₁₄N₂Na₂ | 372.24 | 15.0 |
| dihydrate) | O_{8.2}H₂O | | |
| Zinc sulfate | ZnSO₄•7H₂O | 287.54 | 4.5 |
| heptahydrate | | | |
| Manganese chloride | MnCl₂•2H₂O | 161.88 | 1.0 |
| dihydrate | | | |
| Copper (II) sulfate | CuSO₄•5H₂O | 249.68 | 0.3 |
| pentahydrate | | | |
| Iron (II) sulfate | FeSO₄•7H₂O | 278.02 | 3.0 |
| heptahydrate | | | |

TABLE 6

| | | Lactic | | | | Residual Sugar | | | Peak | | Final |
|---|---|---|---|---|---|---|---|---|---|---|---|
| | | Rate | Overall Lactic | Final Lactic | Final Arabitol | (sucrose, glucose, | Production | Final | Rate | Average | Offline |
| Tank | Strain | (g/Lh) | Yield (g/g) Mass | Titer (g/L) | Titer (g/L) | and fructose) (g/L) | Time (h) | pH | (g/Lh) | OUR | pH |
| EB09 | 1.3 | 2.93 | 0.84 | 126.58 | 1.96 | 1.47 | 43.25 | 2.95 | 4.93 | 12.83 | 2.95 |
| EB11 | 1.3 | 2.95 | 0.84 | 126.15 | 2.11 | 1.40 | 42.75 | 2.95 | 4.95 | 12.95 | 2.96 |
| EB15 | 1.5a | 3.13 | 0.86 | 123.15 | 2.11 | 7.65 | 39.33 | 2.92 | 8.47 | 12.76 | 2.95 |
| EB16 | 1.5a | 3.19 | 0.85 | 124.25 | 2.13 | 4.61 | 39.00 | 2.95 | 9.58 | 12.94 | 2.96 |

### Example 3: Fermentations with Lactate Producing Strains 1.3 and 1.5a at Higher OUR

The lactate producing yeast strains 1.3 and 1.5a were run in fermenters to assess glucose and fructose consumption as well as lactate production. Strains 1.3 and 1.5a were streaked out for single colonies on a YPD plate (bacteriological peptone 20 g/L, yeast extract 10 g/L, glucose 20 g/L, and agar 15 g/L) and incubated until single colonies were visible. Incubation at room temperature resulted in single colony growth in about 72 hours and incubation at 30° C. resulted in single colony growth in about 18 hours. Cells from plates were scraped into sterile seed medium (Table 2.1) and the optical density (OD₆₀₀) was measured. Optical density is measured at a wavelength of 600 nm with a 1 cm path length cuvette using a model Genesys20 spectrophotometer (Thermo Scientific). Seed cultures are run in 500 mL shake flasks containing 50 mL seed medium at 34° C. and 250 RPM until they reach an OD₆₀₀ of 4-8.

A 250 ml capacity fermenter was inoculated with the cell slurry to reach on initial OD600 of 0.1. Separate fermenters are inoculated with the cell slurry for each of the strains, in triplicate. Immediately prior to inoculating, 0.2 L of fermentation medium is added to the fermenter. The fermentation medium is sterilized, pH to 5.5, and contains the components outlined in Table 7.

pH in the fermenters is controlled using MES buffer and 30% calcium carbonate (CaCO₃). The 30% CaCO₃ solution is added before inoculation and at hour 2 for a target pH of 3.0. After the hour 2 addition of CaCO₃ the pH is allowed to freefall and no further pH control occurs. The fermenters are sparged with 1 SLPM (standard liters per minute) air through a sparge ring at the base of the vessel. An oxygen uptake rate of 12-13 mmol O₂/(L*h) or 15-17 mmol O₂/(L*h) is achieved by selecting an appropriate agitation speed, 600±25 RPM or 625±25 RPM respectively. These fermentations are operated such that after the cells achieve a sufficient density, oxygen limitation is achieved and subsequently maintained throughout the rest of the fermentation (e.g., dissolved oxygen less than about 10.) Dissolved oxygen is measured using Mettler Toledo InProR® 6800 sensor (Mettler-Toledo GmbH, Urdorf, Switzerland), calibrated prior to inoculation. 0% is calibrated by unplugging the probe and measuring a null signal, 100% is calibrated using air sparging according to the run conditions in the vessel as detailed above (prior to inoculation). Oxygen uptake rate (“OUR”) is calculated using methods known to those in the art as described above. For this example, Oxygen, N₂ and CO₂ values are measured by a mass spectrometer.

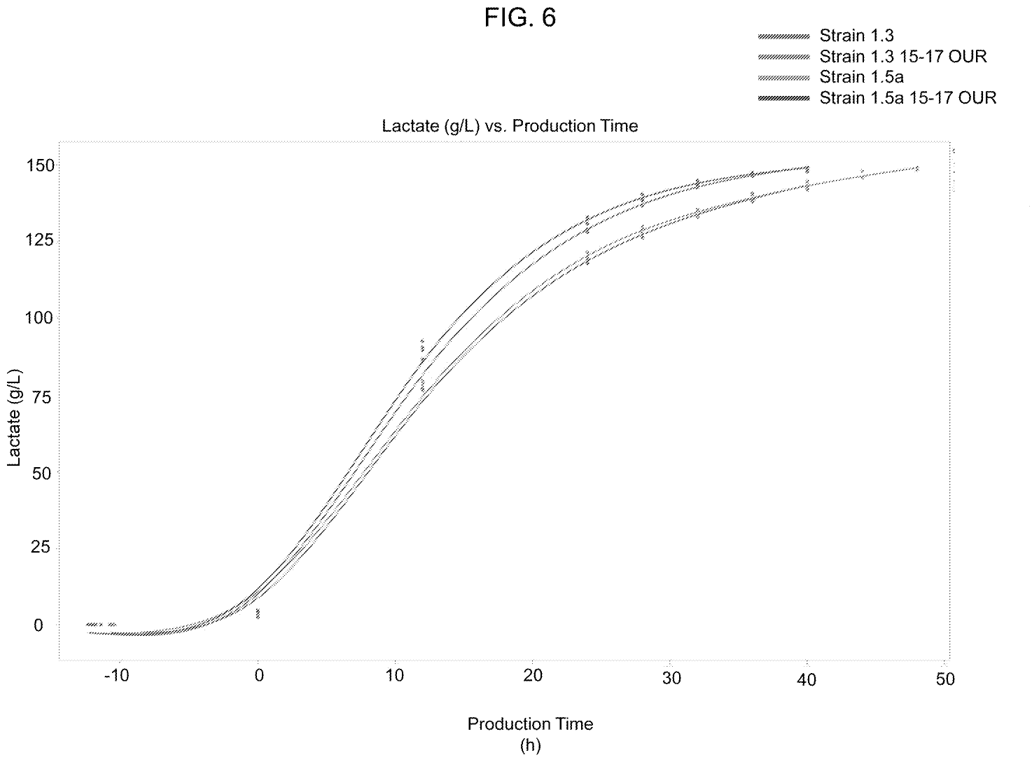

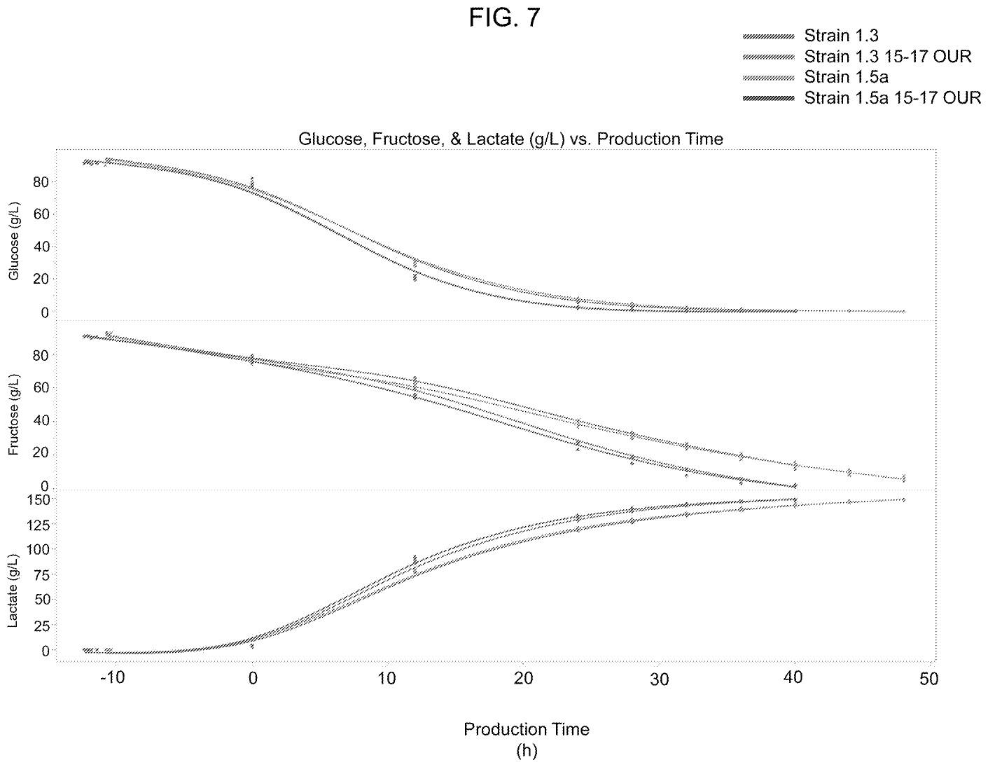

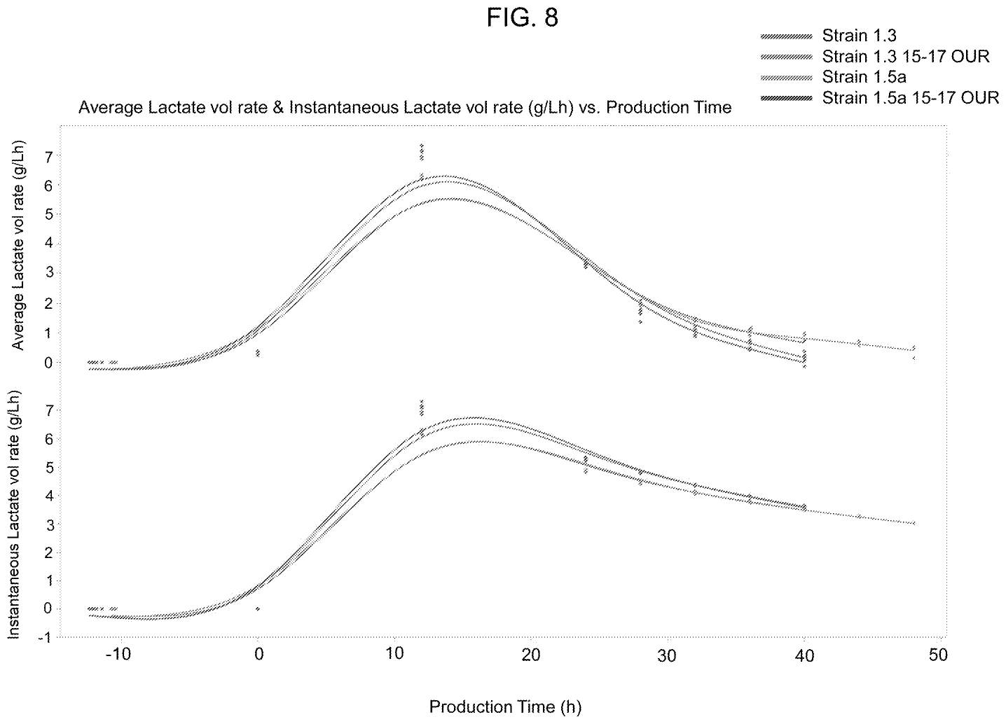

Samples are taken immediately after inoculation, at the end of the batch, and periodically throughout the fermentation. Samples are analyzed for lactate, fructose, sucrose, arabitol, and glucose concentration by high performance liquid chromatography with refractive index detector. Fermentation results are reported in Table 8 and in FIGS. 6-8.

Overall, increasing the OUR to 15-17 in both strains 1.5a and 1.3 resulted in a higher rate of lactate production, a decrease in total production time, and an increase in the peak rate of lactate production compared to the same strains run at an OUR of 12-13.

TABLE 7
Fermentation Medium

| Component | Concentration | |
|---|---|---|
| Sucrose | 180 | g/L |
| Lubrizol 1:100 | 1.07 | mL/L |
| Glycerol 10% | 1.07 | mL/L |
| APK | | |
| (11.7% (w/v) anhydrous KOH, | 1.15 | mL/L |
| 30.3% (w/v) anhydrous phosphoric acid, | | |
| 4.86% (w/v) anhydrous ammonia) | | |
| 50% Urea | 1.72 | mL/L |
| Magnesium Sulfate | 1.00 | mL/L |
| (1000× stock solution at 125 g/L) | | |
| Trace Composition (Table 5) | 1.00 | mL/L |
| D-Biotin Stock Solution | 1.00 | mL/L |
| (1000×, 1.04 g/mL) | | |

TABLE 8
Residual Sugar

| | | Lactic | | | | Residual Sugar | | | Peak | | |
|---|---|---|---|---|---|---|---|---|---|---|---|
| | | Rate | Overall Lactic | Final Lactic | Final Arabitol | (sucrose, glucose, | Production | Final | Rate | Average | Arabitol/ |
| OUR | Strain | (g/Lh) | Yield (g/g) Mass | Titer (g/L) | Titer (g/L) | and fructose) (g/L) | Time (h) | pH | (g/Lh) | OUR | Lactic |
| 12-13 | 1.3 | 3.10 | 0.78 | 148.86 | 3.03 | 5.47 | 48.03 | 3.22 | 6.17 | 11.50 | 0.02 |
| 15-17 | 1.3 | 4.07 | 0.77 | 146.59 | 3.29 | 5.47 | 36.02 | 3.24 | 6.91 | 16.78 | 0.02 |
| 12-13 | 1.5a | 3.09 | 0.79 | 148.57 | 2.98 | 5.94 | 48.02 | 3.24 | 6.27 | 11.64 | 0.02 |
| 15-17 | 1.5a | 4.08 | 0.76 | 147.16 | 3.31 | 4.82 | 36.03 | 3.22 | 7.18 | 16.52 | 0.02 |

### Example 4: Shake Flask Cultures of Strains 1.3, 1.6, 1.7, 1.8a-1.8c, 1.9a-1.9c, and 1.10a-1.10c

The lactate producing yeast strains 1.3, 1.6, 1.7, 1.8a-1.8c, 1.9a-1.9c, and 1.10a-1.10c were run in shake flasks to assess glucose and fructose consumption as well as lactate production. Strains 1.3, 1.6, 1.7, 1.8a-1.8c, 1.9a-1.9c, and 1.10a-1.10c were streaked out for single colonies on YPD plates (bacteriological peptone 20 g/L, yeast extract 10 g/L, glucose 20 g/L, and agar 15 g/L) and incubated until single colonies were visible. Incubation at room temperature resulted in single colony growth in about 72 hours and incubation at 30° C. resulted in single colony growth in about 18 hours. Cells from plates were scraped into production medium (Table 9) and the optical density (OD₆₀₀) was measured. Optical density is measured at a wavelength of 600 nm with a 1 cm path length cuvette using a model Genesys20 spectrophotometer (Thermo Scientific).

A 250 mL non-baffled shake flask containing 0.26 g CaCO₃ was inoculated with the production medium culture to reach an initial OD₆₀₀ of 0.2. Immediately prior to inoculation, 18 mL of the production medium was added to the 250 mL shake flask. The production medium is sterilized, pH to 5.5, and contains the components outlined in Table 9. After inoculation, shake flasks are incubated at 34° C. with a relative humidity of 70% and shaking at 150 rpm for approximately 67 hours.

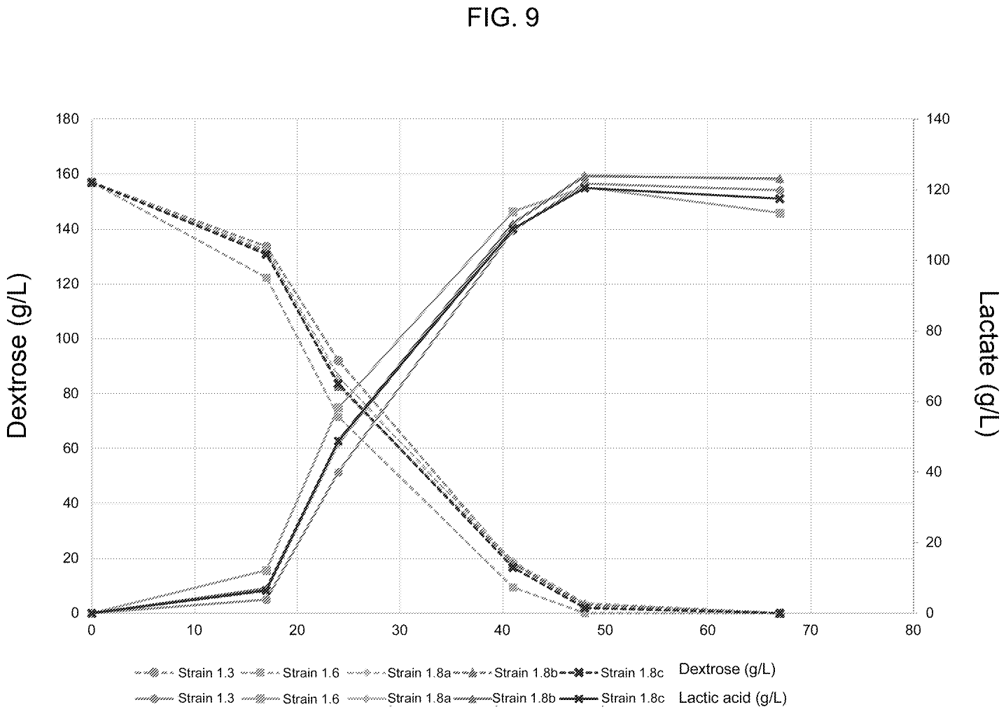

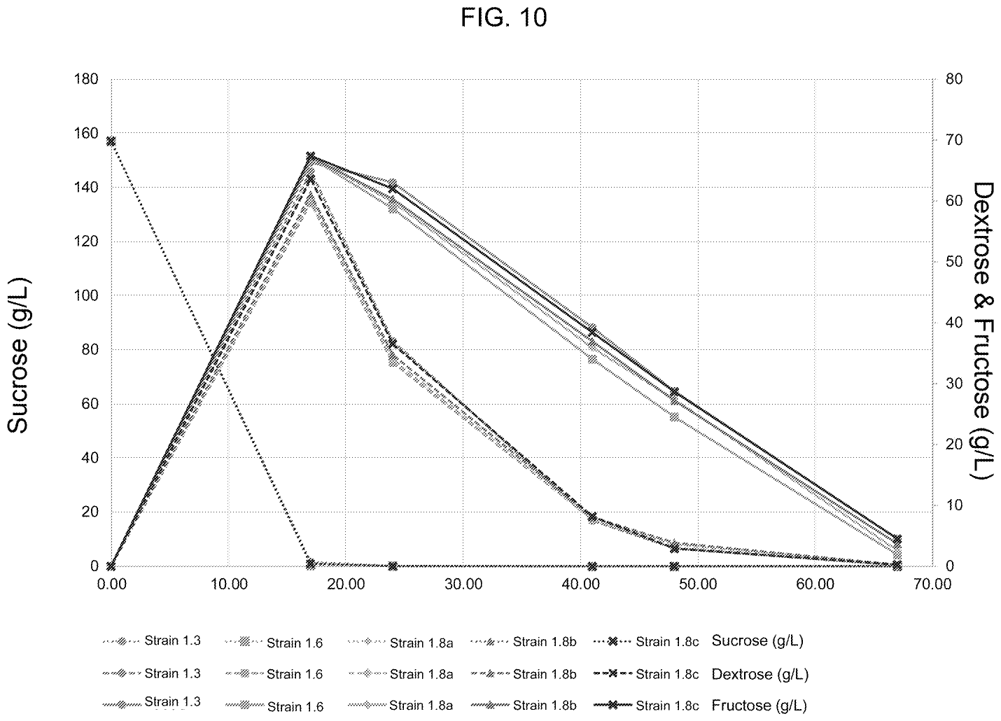

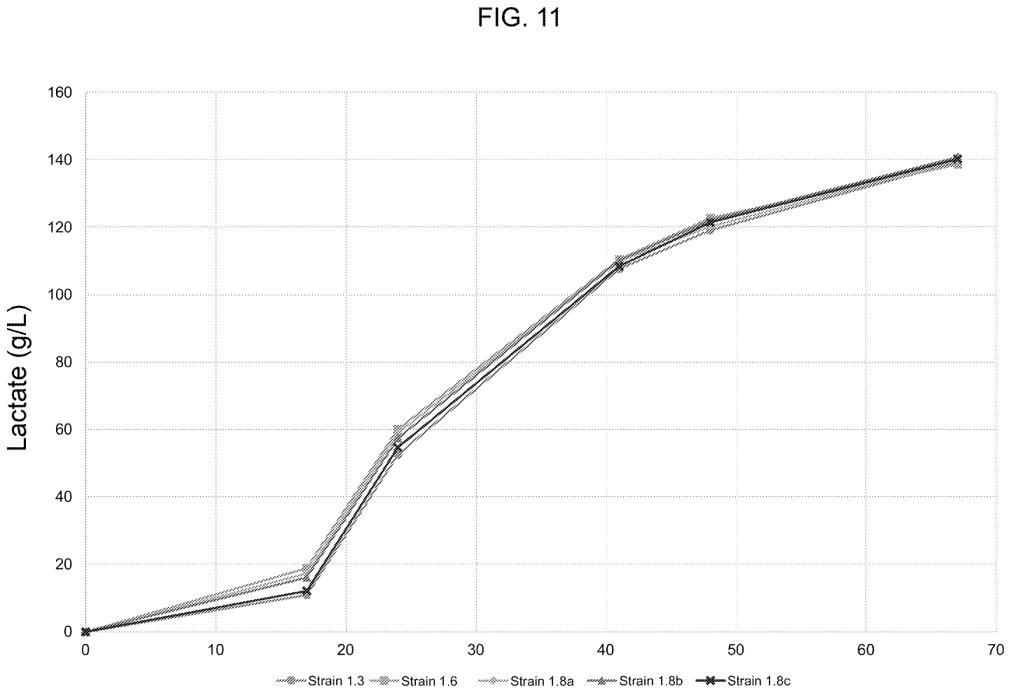

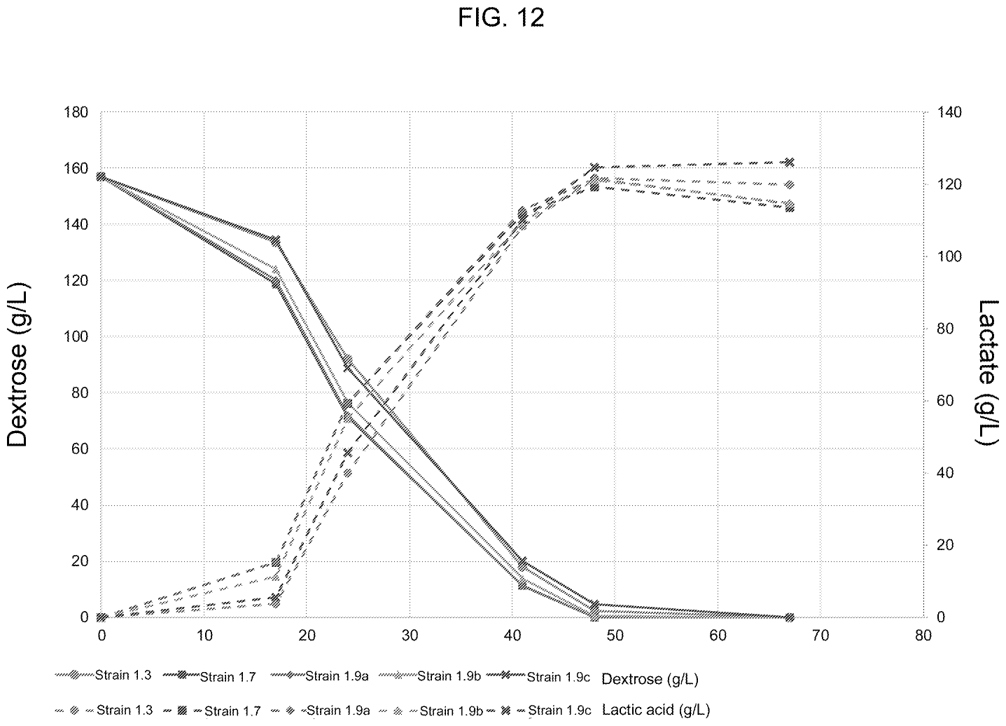

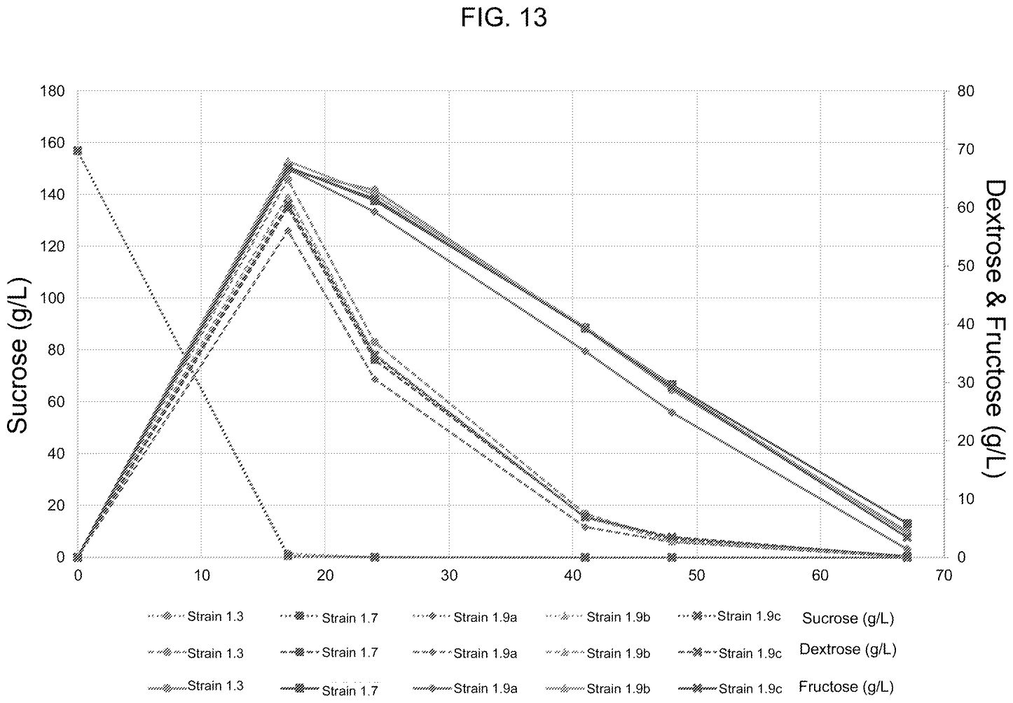

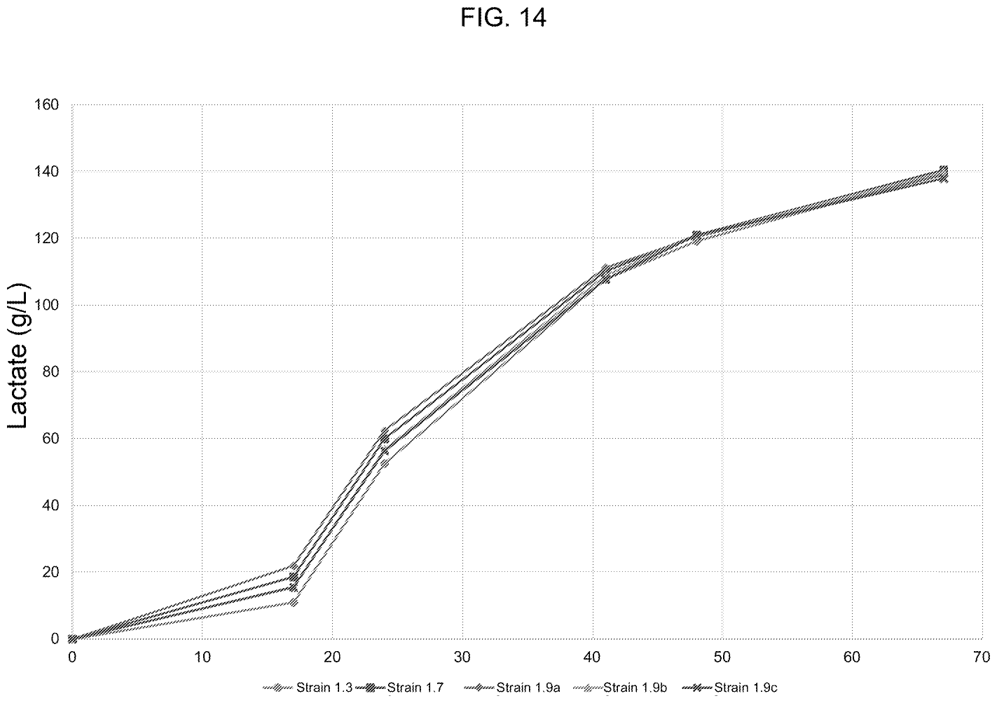

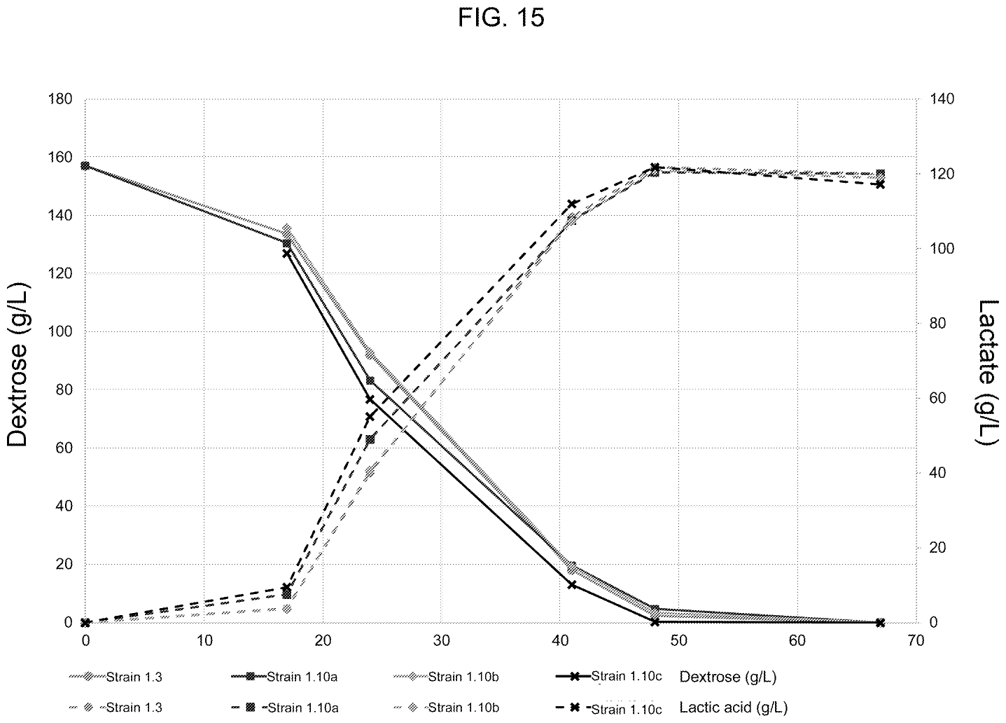

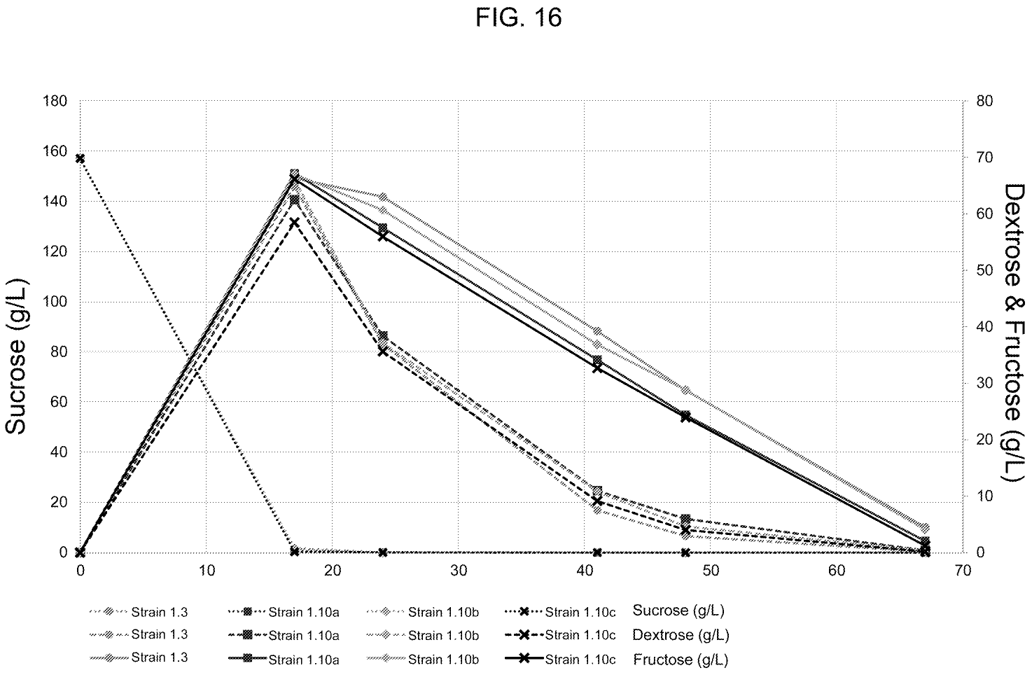

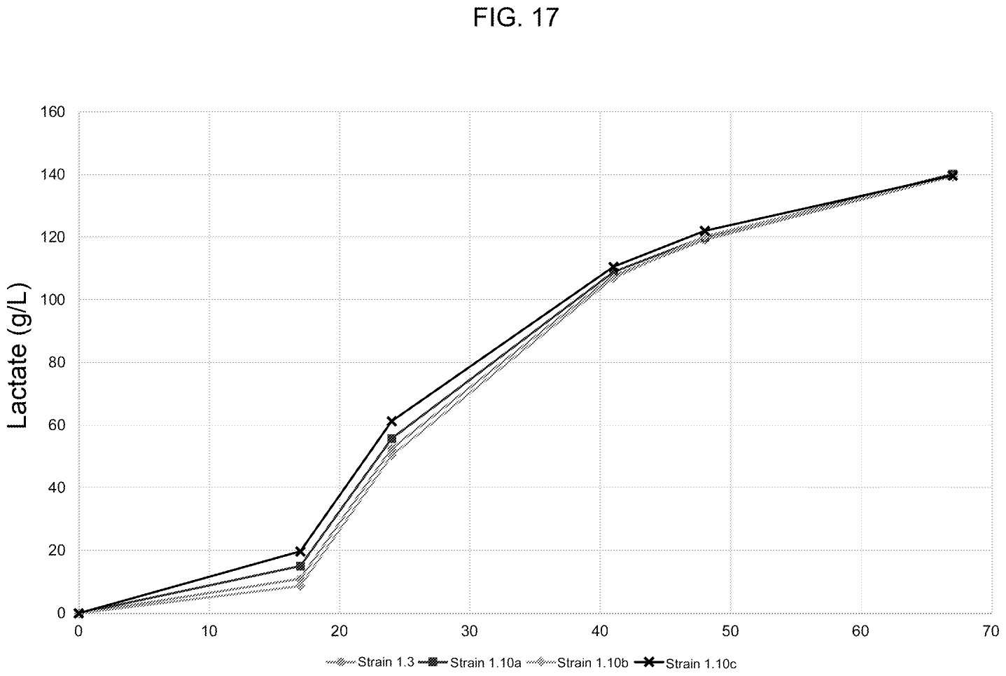

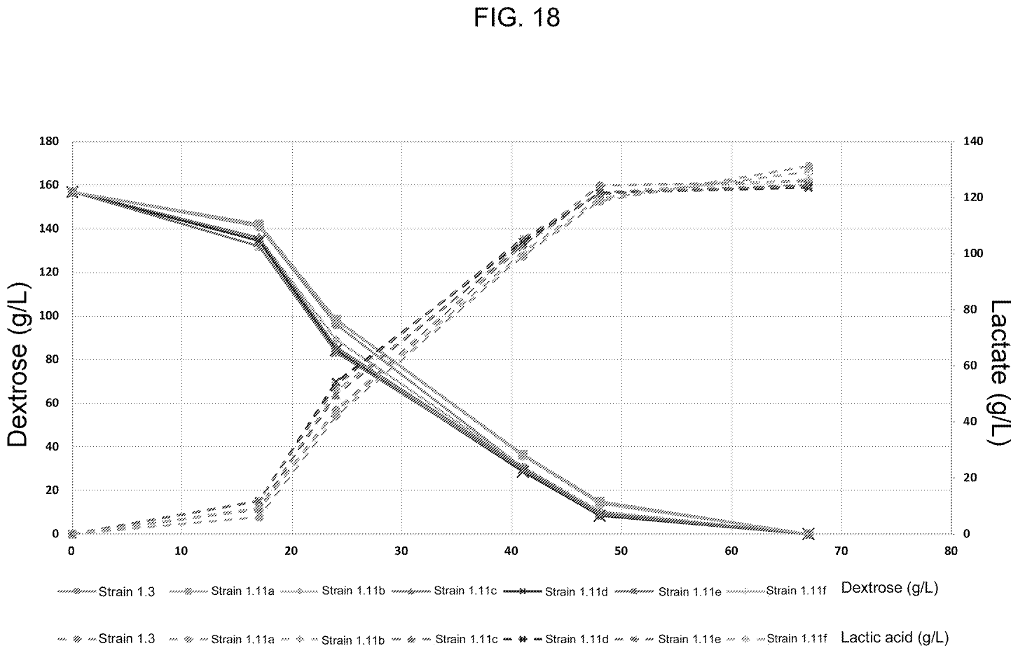

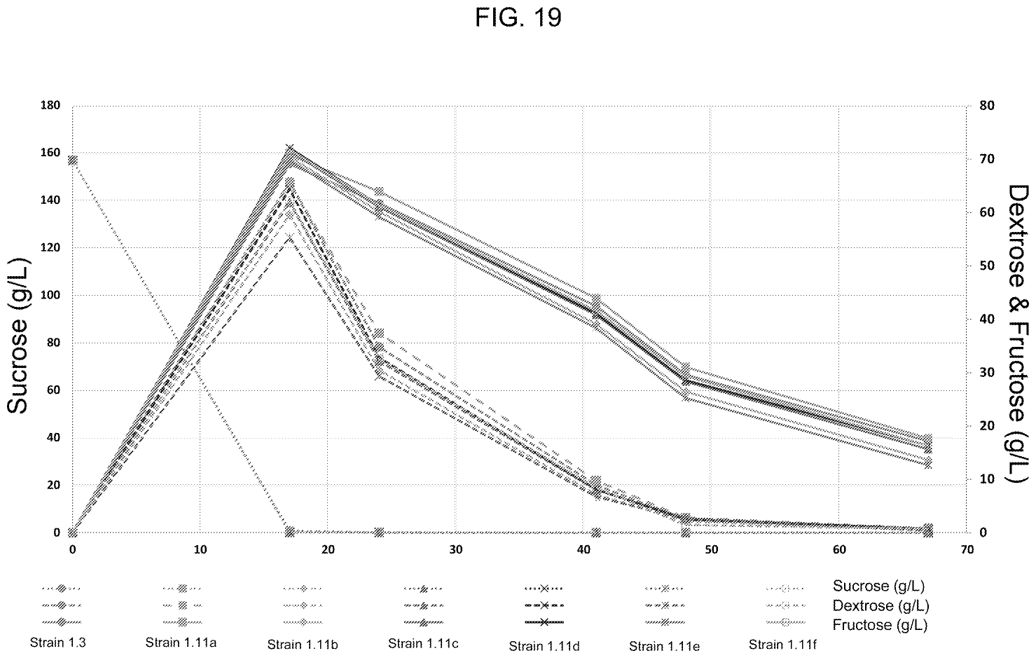

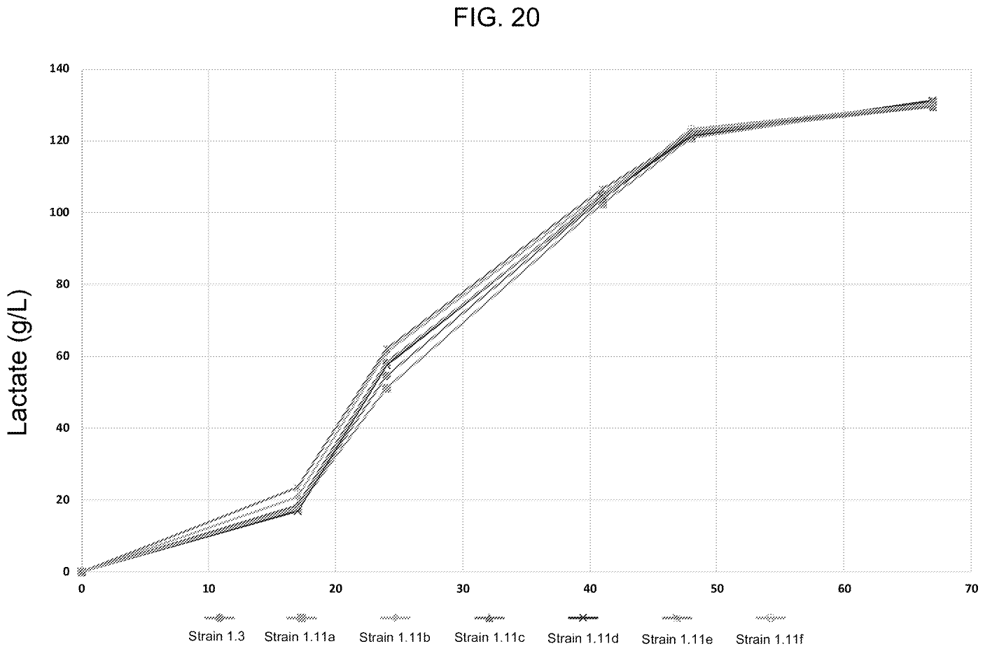

Samples are taken immediately after inoculation, at the end of the batch, and periodically throughout the fermentation. Samples are analyzed for lactate, glucose, fructose, sucrose and arabitol concentration by high performance liquid chromatography with refractive index detector. Fermentation results are reported in FIGS. 9-20.

Overall, strains 1.6, 1.8a, 1.8b, 1.8c, 1.9a, 1.10a, 1.10b, 1.10c, 1.11b, 1.11c, 1.11d, 1.11e, and 1.11f showed an improved rate of fructose consumption relative to the control strain 1.3. Similarly, strains 1.6, 1.7, 1.8a, 1.8b, 1.8c, 1.9a, 1.9b, 1.10a, 1.10b, 1.11b, 1.11c, 1.11d, and 1.11e had higher rates of dextrose consumption and lactate production, especially early in the fermentation, as compared to the control strain 1.3.

TABLE 9
Production Medium

| Component | Concentration | |
|---|---|---|
| Sucrose | 162.75 | g/L |
| Lubrizol 1:100 | 1.07 | mL/L |
| Glycerol 10% | 1.07 | mL/L |
| APK | 1.15 | mL/L |
| (11.7% (w/v) anhydrous KOH, | | |
| 30.3% (w/v) anhydrous phosphoric acid, | | |
| 4.86% (w/v) anhydrous ammonia) | | |
| 50% Urea | 1.72 | mL/L |
| Magnesium Sulfate | 1.00 | mL/L |
| (1000× stock solution at 125 g/L) | | |
| Trace Composition (Table 5) | 1.00 | mL/L |
| D-Biotin Stock Solution | 1.00 | mL/L |
| (1000×, 1.04 g/mL) | | |

1. A genetically engineered yeast cell capable of producing lactate from sucrose, the engineered yeast cell comprising
a polynucleotide encoding an exogenous lactate dehydrogenase enzyme;
a polynucleotide encoding an exogenous invertase enzyme comprising a sequence at least 80% identical to at least one of SEQ ID NOs: 12, 33, 34, and 35;
a deletion or disruption of a native pyruvate decarboxylase (PDC) gene; and
a genetic modification resulting in overexpression of a native hexokinase gene.

2. The engineered yeast cell of claim 1, wherein hexokinase activity in the engineered yeast cell is higher than hexokinase activity in an equivalent yeast cell lacking the genetic modification.

3. The engineered yeast cell of claim 1, wherein peak lactate production rate in the engineered yeast cell, when used in a fermentation process in the presence of sucrose, is higher than peak lactate production rate of an equivalent yeast cell lacking the genetic modification.

4. (canceled)

5. (canceled)

6. The engineered yeast cell of claim 1, wherein the genetic modification comprises addition of an exogenous polynucleotide encoding the native hexokinase such that the genetically engineered yeast cell comprises at least one additional copy of a sequence encoding the native hexokinase.

7. The engineered yeast cell of claim 1, wherein the native hexokinase comprises a sequence at least 80% identical to at least one of SEQ ID NOs: 24 and 25.

8. The engineered yeast cell of claim 1, wherein the engineered yeast cell comprises a deletion or disruption of a glycerol-3-phosphate dehydrogenase (GPD) gene.

9. The engineered yeast cell of claim 1, wherein the engineered yeast cell comprises a deletion or disruption of an L-lactate:cytochrome c oxidoreductase (CYB2) gene.

10. (canceled)

11. (canceled)

12. (canceled)

13. The engineered yeast cell of claim 1, wherein the yeast cell is Crabtree negative.

14. The engineered yeast cell of claim 1, wherein the yeast cell is a yeast of the Issatchenkia orientalis/Pichia fermentans clade.

15. The engineered yeast cell of claim 1, wherein the yeast cell is an Issatchenkia orientalis cell.

16. (canceled)

17. The engineered yeast cell of claim 1, wherein the yeast cell additionally comprises an exogenous polynucleotide encoding a fructokinase.

18. The engineered yeast cell of claim 17, wherein the fructokinase comprises a sequence at least 80% identical to SEQ ID NO: 26.

19. (canceled)

20. (canceled)

21. The engineered cell of claim 1, wherein the lactate dehydrogenase comprises a sequence a sequence at least 80% identical to at least one of SEQ ID NOs: 30 and 31.

22. (canceled)

23. (canceled)

24. A method for producing lactate from sucrose, the method comprising:
contacting a substrate comprising sucrose with the engineered yeast cell of claim 1, wherein fermentation of the substrate by the engineered yeast produces lactate.

25. The method of claim 24, wherein the volumetric oxygen uptake rate (OUR) is 0.5 to 40 mmol O₂/(L·h).

26. (canceled)

27. The method of claim 24, wherein peak lactate production rate is at least 5 g L⁻¹ h⁻.

28. The method of claim 24, wherein lactate is produced at a rate of at least 1.5 g L⁻¹ h⁻.

29. The method of claim 24, wherein the fermentation temperature is in the range of 20° C. to 45° C.

30. The method of claim 24, wherein the lactate titer is at least 30, at least 80, at least 100, or at least 120 g/L.

31. A method for producing lactate from sucrose, the method comprising:
contacting a substrate comprising sucrose with an engineered yeast cell comprising
a polynucleotide encoding an exogenous lactate dehydrogenase enzyme;
a polynucleotide encoding an exogenous invertase enzyme;
a deletion or disruption of a native pyruvate decarboxylase (PDC) gene; and
a genetic modification resulting in overexpression of a native hexokinase gene,
wherein fermentation of the substrate by the engineered yeast produces lactate.

32-37. (canceled)

38. A genetically engineered Issatchenkia orientalis cell capable of producing lactate from sucrose, the engineered yeast cell comprising
a polynucleotide encoding an exogenous lactate dehydrogenase enzyme;
a polynucleotide encoding an exogenous invertase enzyme;
a deletion or disruption of a native pyruvate decarboxylase (PDC) gene; and
a genetic modification resulting in overexpression of a native hexokinase gene, wherein the engineered I. orientalis cell is capable of producing lactate at a titer of at least 30.
